(19) United States
(12) Patent Application Publication
(10) Pub. No.: US 2020/0383961 A1
(43) Pub. Date: Dec. 10, 2020

(54) TRIPLET THERAPIES OF HDAC INHIBITORS, PD-L1 AND/OR PD-1 INHIBITORS, AND CTLA-4 INHIBITORS

(71) Applicant: HUYA Bioscience International, LLC, San Diego, CA (US)

(72) Inventors: Mireille GILLINGS, Las Vegas, NV (US); Reid BISSONNETTE, Carlsbad, CA (US)

(21) Appl. No.: 16/894,532

(22) Filed: Jun. 5, 2020

Related U.S. Application Data
(60) Provisional application No. 62/858,317, filed on Jun. 6, 2019.

(51) Int. Cl. A61K 31/4406; A61K 39/395; A61K 9/00; A61K 45/06

(52) U.S. Cl. CPC A61K 31/4406; A61K 45/06; A61K 9/0019; A61K 39/3955

(57) ABSTRACT

Provided herein is a combination therapy comprising an HDAC inhibitor (HDACi) a PD-L1 and/or a PD-1 inhibitor, and a CTLA-4 inhibitor. The combination therapy provided herein can be a kit or the composition or a pharmaceutical composition. Also, provided herein is a method of treating cancer using the combination therapy.

### CROSS-REFERENCE

This application claims benefit of U.S. Provisional Application No. 62/858,317, filed on Jun. 6, 2019, which is herein incorporated by reference in its entirety.

### FIELD

The present invention relates to combinations of HDAC inhibitors, PD-1 inhibitors, PD-L1 inhibitors, and CTLA-4 inhibitors, among other checkpoint inhibitors, and the use of such combinations in the treatment of cancer.

### BACKGROUND OF THE INVENTION

Cancer is a significant cause of morbidity and mortality worldwide. While the standards of care for many different cancer types have greatly improved over the years, current standards of care still fail to meet the need for effective therapies to improve treatment of cancer. The clinical use of immuno-oncology agents targeting cytotoxic T-lymphocyte-associated protein 4 (CTLA-4) and the programmed cell death receptor-1 (PD-1) and its ligand PD-L1, have resulted in improvements over the standard of care in the treatment of many cancer types. While these checkpoint inhibitors have produced improved clinical responses in such certain cancers, durable clinical responses only occur in approximately 10-45% of patients. Moreover, a significant number of tumors are either resistant or become refractory. Epigenetic modifiers such as histone deacetylase inhibitors (HDACi) have been successful in the treatment of some hematologic malignancies, but despite preclinical data demonstrating activity against solid tumors, this result has not translated to the clinic as a monotherapy. Accordingly, there is a need in the art for new therapies, including, for example, combination therapies for the treatment of cancers. Provided herein are solutions to these and other problems in the art.

### SUMMARY OF THE INVENTION

Provided herein, inter alia, are combinations that include an HDAC inhibitor (HDACi) and a PD-L1 and/or PD-1 inhibitor, further in combination with a CTLA-4 inhibitor. The combinations include a compound of formula I and a PD-L1 and/or PD-1 inhibitor, further in combination with a CTLA-4 inhibitor. In certain instances, the PD-L1 inhibitor, PD-1 inhibitor, and/or CTLA-4 inhibitor are antibodies. In some embodiments, the combination is an HDAC inhibitor (HDACi) a PD-L1 inhibitor, and a CTLA-4 inhibitor. In some embodiments, the combination is an HDAC inhibitor (HDACi) a PD-1 inhibitor, and a CTLA-4 inhibitor.

In a first aspect of the disclosure provided herein is a combination comprising a therapeutically effective amount of a PD-L1 inhibitor, a PD-1 inhibitor, a CTLA-4 inhibitor, a CD276 inhibitor, a therapeutically effective amount of a compound of formula I, or any combination thereof, wherein formula I is:

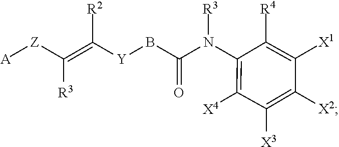

wherein, A is phenyl or a heterocyclic group, optionally substituted with 1 to 4 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkythio, C₁-C₄ perfluoroalkyl, C₁-C₄ perfluoroalkyloxy, C₁-C₄ alkoxycarbonyl, phenyl, and a heterocyclic group; B is phenyl optionally substituted with 1 to 3 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, C₁-C₄ alkoxycarbonyl, and phenyl; Y is a moiety comprising —CO— which is linear and in which the distances between the centroid of ring B (W1), the centroid of ring A (W2) and an oxygen atom as a hydrogen bond acceptor in the moiety Y (W3) are: W1-W2=about 6.0 Å, W1-W3=about 3.0 Å to about 6.0 Å, and W2-W3=about 4.0 Å to about 8.0 Å, respectively; Z is a bond or C₁-C₄ alkylene, —O—, —S—, —NH—, —CO—, —CS—, —SO—, or —SO₂—; R¹ and R² are independently hydrogen or C₁-C₄ alkyl; R³ is hydrogen or C₁-C₄ alkyl; R⁴ is hydrogen or —NH₂; one of X¹, X², X³, or X⁴ is halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, or C₁-C₄ alkoxycarbonyl optionally substituted with halogen or C₁-C₄ alkyl, while the others of X¹, X², X³, or X⁴ are independently hydrogen, provided, however, that when R⁴ is hydrogen, one of X¹, X², X³, or X⁴ is —NH₂, an aminoalkyl group or an alkylamino group. In some embodiments, said compound of formula I has the structure of formula Ia:

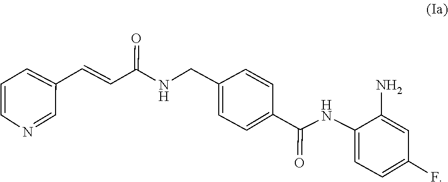 [SMILES: Nc1cc(F)ccc1NC(=O)c1ccc(CNC(=O)/C=C/c2cccnc2)cc1]

In some embodiments, said compound of formula I is N-(2-amino-4-fluorophenyl)-4-[[[(2E)-1-oxo-3-(3-pyridinyl)-2-propen-1-yl]amino]methyl]benzamide. In some embodiments, said PD-L1 inhibitor, PD-1 inhibitor, CTLA-4 inhibitor, and/or CD276 inhibitor is a small molecule compound, a nucleic acid, a peptide, a protein, an antibody, a peptibody, a diabody, a minibody, a single-chain variable fragment (ScFv), or a fragment or variant thereof. In some embodiments, at least one of said PD-L1 inhibitor, PD-1 inhibitor, CTLA-4 inhibitor, and/or CD276 inhibitor is an antibody. In some embodiments, said inhibitor antibody is a monoclonal antibody. In some embodiments, said inhibitor antibody comprises a human antibody, a mouse antibody, a chimeric antibody, a humanized antibody, or a chimeric humanized antibody. In some embodiments, said inhibitor antibody is a human antibody or a humanized antibody. In some embodiments, said inhibitor antibody is present at an amount of about 0.1 mg/kg to about 30 mg/kg. In some embodiments, said inhibitor antibody is present at an amount of about 0.5 mg/kg to about 15 mg/kg. In some embodiments, said inhibitor antibody is present at an amount of about: 0.1 mg/kg, 0.3 mg/kg, 1 mg/kg, 2 mg/kg, 2.5 mg/kg, 3 mg/kg, 5 mg/kg, 10 mg/kg, or 20 mg/kg. In some embodiments, said combination is suitable for parenteral administration to a cancer patient. In some embodiments, said parenteral administration comprises intravenous (IV) administration.

Another aspect of the present disclosure comprises a pharmaceutical composition comprising a combination of any one of the embodiments described herein, and a pharmaceutically acceptable excipient.

Another aspect of the present disclosure comprises a kit comprising the combination of any of one of the embodiments described herein or a pharmaceutical composition of the embodiments described herein. In some embodiments, the kit further comprises at least one administration device. In some embodiments, components in the kit are sterilized.

Another aspect of the present disclosure comprises a method for treating cancer, said method comprising administering a therapeutically effective amount of a combination of any one of the embodiments described herein or a pharmaceutical composition of the embodiments described herein to a subject in need thereof. In some embodiments, said subject has a mutated BRAF gene. In some embodiments, said cancer is a solid tumor cancer selected from the group consisting of squamous cell carcinoma, nonsquamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, colorectal cancer, pancreatic cancer, lymphoma, leiomyosarcoma, liposarcoma, synovial sarcoma, or malignant peripheral sheath tumor (MPNST). In some embodiments, said cancer is non-small cell lung cancer (NSCLC), hepatocellular carcinoma, melanoma, ovarian cancer, breast cancer, pancreatic cancer, renal cell carcinoma, or colorectal cancer. In some embodiments, said cancer is lymphoma, Non-Hodgkin's lymphoma (NHL), Hodgkin's Lymphoma, Reed-Sternberg disease, multiple myeloma (MM), acute myelogenous leukemia (AML), chronic myelogenous leukemia (CML), acute lymphocytic leukemia, (ALL), or chronic lymphocytic leukemia (CLL). In some embodiments, said cancer patient is treatment naïve. In some embodiments, said cancer patient is treatment naïve for non-small cell lung cancer (NSCLC), hepatocellular carcinoma, melanoma, ovarian cancer, breast cancer, pancreatic cancer, renal cell carcinoma, or colorectal cancer. In some embodiments, said combination is administered to said cancer patient as a first line therapy. In some embodiments, said combination is administered to said cancer patient as a second, third, fourth, fifth, or sixth line of treatment. In some embodiments, said combination is administered to said cancer patient following treatment with at least one anti-cancer therapy. In some embodiments, said anti-cancer therapy comprises chemotherapy, radiotherapy, surgery, targeted therapy, immunotherapy, or a combination thereof. In some embodiments, said cancer is resistant to at least one anti-cancer agent. In some embodiments, said compound of formula I and said inhibitor of said combination are administered simultaneously or sequentially. In some embodiments, said compound of formula I is administered 2 to 3 times per week. In some embodiments, said compound of formula I is administered daily. In some embodiments, said PD-L1 inhibitor, PD-1 inhibitor, CTLA-4 inhibitor, and/or CD276 inhibitor and said compound of formula I are concomitantly administered on day 1 of an administration regimen. In some embodiments, said combination is administered to said patient as a regimen. In some embodiments, said regimen is repeated until disease progression or unacceptable toxicity. In some embodiments, said regimen comprises a rest period of at least 1 day between consecutive administration periods. In some embodiments, said compound of formula I of said combination is administered 2 to 3 times per week in said regimen and said PD-L1 inhibitor, PD-1 inhibitor, CTLA-4 inhibitor, and/or CD276 inhibitor is administered every 2 to 3 weeks. In some embodiments, said compound of formula I of said combination is administered once a day (“QD”) for 21 days in said regimen and said inhibitor antibody is administered every 2 to 3 weeks. In some embodiments, said method of treating cancer inhibits metastasis of said cancer in said patient. In some embodiments, said method of treating cancer reduces tumor or tumor burden in said patient. In some embodiments, said method of treating cancer inhibits pre-existing metastasis of said cancer in said patient. In some embodiments, said method of treating cancer prolongs the time to disease progression of said cancer in said patient. In some embodiments, said method of treating cancer prolongs the survival of said patient. In some embodiments, said method of treating cancer increases progression-free survival of said patient.

Another aspect of the present disclosure comprises a method for treating cancer comprising administering a therapeutically effective amount of a combination of a histone deacetylase inhibitor (HDAC inhibitor) and a PD-L1 inhibitor and/or a PD-1 inhibitor, plus a CTLA-4 inhibitor, to a subject in need of treatment and whose cancer has been previously treated with a checkpoint inhibitor. A method for treating cancer comprising administering a therapeutically effective amount of a PD-L1 inhibitor, a PD-1 inhibitor, a CTLA-4 inhibitor, a CD276 inhibitor, a histone deacetylase inhibitor (HDAC inhibitor), or any combination thereof, to a subject in need of treatment and whose cancer has been previously treated with a checkpoint inhibitor.

Another aspect of the present disclosure comprises a method for treating cancer comprising administering a therapeutically effective amount of a PD-L1 inhibitor, a PD-1 inhibitor, a CTLA-4 inhibitor, a CD276 inhibitor, a histone deacetylase inhibitor (HDAC inhibitor), or any combination thereof, to a subject in need of treatment wherein said subject comprises a mutated BRAF gene. A method for treating cancer comprising administering a therapeutically effective amount of: a compound of formula I, wherein formula I is:

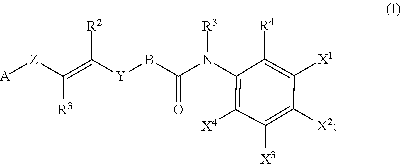

wherein, A is phenyl or a heterocyclic group, optionally substituted with 1 to 4 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkythio, C₁-C₄ perfluoroalkyl, C₁-C₄ perfluoroalkyloxy, C₁-C₄ alkoxycarbonyl, phenyl, and a heterocyclic group; B is phenyl optionally substituted with 1 to 3 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, C₁-C₄ alkoxycarbonyl, and phenyl; Y is a moiety comprising —CO— which is linear and in which the distances between the centroid of ring B (W1), the centroid of ring A (W2) and an oxygen atom as a hydrogen bond acceptor in the moiety Y (W3) are: W1-W2=about 6.0 Å, W1-W3=about 3.0 Å to about 6.0 Å, and W2-W3=about 4.0 Å to about 8.0 Å, respectively; Z is a bond or C₁-C₄ alkylene, —O—, —S—, —NH—, —CO—, —CS—, —SO—, or —SO₂—; R¹ and R² are independently hydrogen or C₁-C₄ alkyl; R³ is hydrogen or C₁-C₄ alkyl; R⁴ is hydrogen or —NH₂, one of X¹, X², X³, or X⁴ is halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, or C₁-C₄ alkoxycarbonyl optionally substituted with halogen or C₁-C₄ alkyl, while the others of X¹, X², X³, or X⁴ are independently hydrogen, provided, however, that when R⁴ is hydrogen, one of X¹, X², X³, or X⁴ is —NH₂, an aminoalkyl group or an alkylamino group; and one or more inhibitor antibodies, wherein said one or more inhibitor antibodies comprise a PD-L1 inhibitor, a PD-1 inhibitor, a CTLA-4 inhibitor, a CD276 inhibitor, or any combination thereof, and wherein said one or more inhibitor antibodies are present at an amount of about 0.1 mg/kg to about 30 mg/kg; to a subject in need of treatment. In some embodiments, said compound of formula I has the structure of formula Ia:

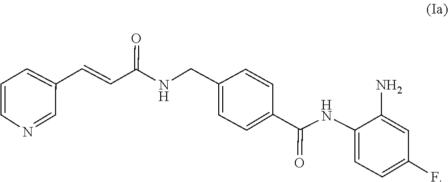 [SMILES: Nc1cc(F)ccc1NC(=O)c1ccc(CNC(=O)/C=C/c2cccnc2)cc1]

In some embodiments, said compound of formula I is N-(2-amino-4-fluorophenyl)-4-[[[(2E)-1-oxo-3-(3-pyridinyl)-2-propen-1-yl]amino]methyl]benzamide. In some embodiments, said one or more inhibitor antibodies are monoclonal antibodies. In some embodiments, said one or more inhibitor antibodies comprise a human antibody, a mouse antibody, a chimeric antibody, a humanized antibody, or a chimeric humanized antibody. In some embodiments, said inhibitor antibody is a human antibody or a humanized antibody. In some embodiments, said cancer is a solid tumor cancer selected from the group consisting of squamous cell carcinoma, nonsquamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, colorectal cancer, pancreatic cancer, lymphoma, leiomyosarcoma, liposarcoma, synovial sarcoma, or malignant peripheral sheath tumor (MPNST). In some embodiments, said cancer patient is treatment naïve. In some embodiments, said cancer patient is treatment naïve for non-small cell lung cancer (NSCLC), hepatocellular carcinoma, melanoma, ovarian cancer, breast cancer, pancreatic cancer, renal cell carcinoma, or colorectal cancer. In some embodiments, said combination is administered to said cancer patient as a first line therapy. In some embodiments, said combination is administered to said cancer patient as a second, third, fourth, fifth, or sixth line of treatment. In some embodiments, said combination is administered to said cancer patient following treatment with at least one anti-cancer therapy. In some embodiments, said anti-cancer therapy comprises chemotherapy, radiotherapy, surgery, targeted therapy, immunotherapy, or a combination thereof. In some embodiments, said cancer is resistant to at least one anti-cancer agent. In some embodiments, said compound of formula I and said inhibitor of said combination are administered simultaneously or sequentially. In some embodiments, said compound of formula I is administered 2 to 3 times per week. In some embodiments, said compound of formula I is administered daily. In some embodiments, said PD-L1 inhibitor, PD-1 inhibitor, CTLA-4 inhibitor, and/or CD276 inhibitor and said compound of formula I are concomitantly administered on day 1 of an administration regimen. In some embodiments, said PD-L1 inhibitor, PD-1 inhibitor, CTLA-4 inhibitor, and/or CD276 inhibitor and said compound of formula I are administered to said patient as a regimen. In some embodiments, said regimen is repeated until disease progression or unacceptable toxicity. In some embodiments, said regimen comprises a rest period of at least 1 day between consecutive administration periods. In some embodiments, said compound of formula I is administered 2 to 3 times per week and said PD-L1 inhibitor, PD-1 inhibitor, CTLA-4 inhibitor, and/or CD276 inhibitor is administered every 2 to 3 weeks. In some embodiments, said compound of formula I of said combination is administered once a day (“QD”) for 21 days in said regimen and said inhibitor antibody is administered every 2 to 3 weeks.

Another aspect of the present disclosure provides for a combination that includes a therapeutically effective amount of 1) a PD-L1 inhibitor and/or PD-1 inhibitor, 2) a therapeutically effective amount of a CTLA-4 inhibitor, and 3) a therapeutically effective amount of a compound of formula I:

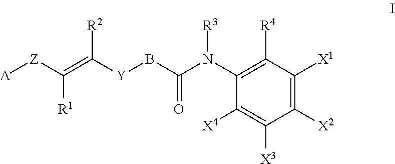

wherein A is phenyl or a heterocyclic group, optionally substituted with 1 to 4 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkythio, C₁-C₄ perfluoroalkyl, C₁-C₄ perfluoroalkyloxy, C₁-C₄ alkoxycarbonyl, phenyl, and a heterocyclic group,

wherein B is phenyl optionally substituted with 1 to 3 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, C₁-C₄ alkoxycarbonyl, and phenyl,

wherein Y is a moiety comprising —CO— which is linear and in which the distances between the centroid of ring B (W1), the centroid of ring A (W2) and an oxygen atom as a hydrogen bond acceptor in the moiety Y (W3) are: W1-W2=about 6.0 Å, W1-W3=about 3.0 Å to about 6.0 Å, and W2-W3=about 4.0 Å to about 8.0 Å, respectively,

wherein Z is a bond or C₁-C₄ alkylene, —O—, —S—, —NH—, —CO—, —CS—, —SO—, or —SO₂—,

wherein R¹ and R² are independently hydrogen or C₁-C₄ alkyl,

wherein R³ is hydrogen or C₁-C₄ alkyl, and

wherein R⁴ is hydrogen or —NH₂; and

wherein one of X¹, X², X³, or X⁴ is halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, or C₁-C₄ alkoxycarbonyl optionally substituted with halogen or C₁-C₄ alkyl, while the others of X¹, X², X³, or X⁴ are independently hydrogen; provided, however, that when R⁴ is hydrogen, one of X¹, X², X³, or X⁴ is —NH₂, an aminoalkyl group, or an alkylamino group.

In one embodiment, the compound of formula I is N-(2-amino-4-fluorophenyl)-4-[[[(2E)-1-oxo-3-(3-pyridinyl)-2-propen-1-yl]amino]methyl]benzamide, referred to herein as HBI-8000, or chidamide.

In another embodiment, the PD-L1 inhibitor is a small molecule compound, a nucleic acid, a peptide, a protein, an antibody, a peptibody, a diabody, a minibody, a single-chain variable fragment (ScFv), or a fragment or variant thereof

In still another embodiment, the PD-L1 inhibitor is an antibody.

In yet another embodiment, the PD-L1 inhibitor antibody is selected from durvalumab, avelumab, atezolizumab, BMS-936559, STI-A1010, STI-A1011, STI-A1012, STI-A1013, STI-A1014, or STI-A1015 (Sorrento Therapeutics).

In another embodiment, the PD-1 inhibitor is a small molecule compound, a nucleic acid, a peptide, a protein, an antibody, a peptibody, a diabody, a minibody, a single-chain variable fragment (ScFv), or a fragment or variant thereof

In still another embodiment, the PD-1 inhibitor is an antibody.

In yet another embodiment, the PD-1 antibody is selected from nivolumab, pembrolizumab, pidilizumab, REGN2810 (also known as SAR-439684), PDR001, SHR-1210 or MEDI0680.

In another embodiment, the CTLA-4 inhibitor is a small molecule compound, a nucleic acid, a peptide, a protein, an antibody, a peptibody, a diabody, a minibody, a single-chain variable fragment (ScFv), or a fragment or variant thereof

In still another embodiment, the CTLA-4 inhibitor is an antibody.

In yet another embodiment, the CTLA-4 antibody is ipilimumab.

In another aspect is a pharmaceutical composition that includes a combination described herein and a pharmaceutically acceptable excipient.

In still another aspect is a kit that includes a combination or a pharmaceutical composition as described herein.

In still another aspect is a method for treating cancer by administering a therapeutically effective amount of a combination or a pharmaceutical composition described herein to a patient in need thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows median tumor volume amongst treatment groups including a combination of compounds of formula I, a CTLA-4 inhibitory antibody, and a PD-1 inhibitory antibody. The dosing of each treatment is indicated by the arrows below the graph.

FIG. 2 shows a Kaplan-Meier survival graph for the same experimental groups from FIG. 1.

FIG. 3A shows the probability of progression free survival (“PFS”) in terms of months resulting from a combination therapy comprising compounds of formula I and nivolumab in melanoma.

FIG. 3B shows the PFS for patients treated with nivolumab monotherapy, ipilimumab monotherapy, or a nivolumab plus ipilimumab combination therapy.

FIG. 4 shows checkpoint inhibitor (“CPI”)-naïve subjects dosed with compounds of formula I in combination with nivolumab.

FIG. 5 shows total time on treatment regime, termination reason, and best ORR for melanoma subjects treated with compounds of formula I and a PD-1 inhibitory antibody.

FIG. 6A shows immune gene activation in response to administration of the compounds of formula I, a PD-1 inhibitory antibody, and a combination of the compounds of formula I and a PD-1 inhibitory antibody.

FIG. 6B shows improvement on survival amongst the experimental group treated with the combination therapy compared to the compounds of formula I alone or the PD-1 inhibitory antibody alone.

FIG. 7A shows an estimated PFS for relapsed or refractory peripheral T-cell lymphoma (“RR/PTCL”) patients given the compounds of formula I were used as a monotherapy.

FIG. 7B shows an estimated survival graph for relapsed or refractory peripheral T-cell lymphoma (“RR/PTCL”) patients given the compounds of formula I were used as a monotherapy.

### DETAILED DESCRIPTION

### Definitions

All patents, applications, published applications and other publications cited herein are incorporated by reference in their entirety. Unless defined otherwise, all technical and scientific terms used herein have the same meaning as commonly understood by those of ordinary skill in the art to which the invention belongs. The chemical structures and formulae set forth herein are constructed according to the standard rules of chemical valency known in the chemical arts. Should a discrepancy exist between a depicted structure and a name given for that structure, the depicted structure is to be accorded more weight. Where the stereochemistry of a structure or a portion of a structure is not indicated in a depicted structure or a portion of the depicted structure, the depicted structure is to be interpreted as encompassing all of its possible stereoisomers.

Any methods, devices and materials similar or equivalent to those described herein can be used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the invention described herein.

The term “PD-L1 inhibitor” refers to a moiety (e.g., compound, nucleic acid, polypeptide, antibody) that decreases, inhibits, blocks, abrogates or interferes with the activity, binding of PD-L1 to its receptor, PD-1, or expression of PD-L1 (e.g., Programmed Cell Death 1 Ligand; PD-L1 (CD274); GI: 30088843), including variants, isoforms, species homologs of human PD-L1 (e.g., mouse) and analogs that have at least one common epitope with PD-L1. A PD-L1 inhibitor includes molecules and macromolecules such as, for example, compounds (small molecule compounds), nucleic acids, polypeptides, antibodies, peptibodies, diabodies, minibodies, single-chain variable fragments (ScFv), and fragments or variants thereof. Thus, a PD-L1 inhibitor as used herein refers to any moiety that antagonizes PD-L1 activity, its binding to PD-1, or its expression. PD-L1 inhibitor efficacy can be measured, for example, by its inhibitor concentration at 50% (half-maximal inhibitor concentration or IC₅₀). PD-L1 inhibitors include exemplary compounds and compositions described herein. A PD-L1 inhibitor antibody refers to a PD-L1 inhibitor which is a monoclonal or polyclonal antibody as described herein.

The terms “durvalumab,” “avelumab,” “atezolizumab,” “BMS-936559,” “STI-A1010,” “STI-A1011,” “STI-A1012,” “STI-A1013,” “STI-A1014,” and “STI-A1015” are used in accordance with their plain and ordinary meaning as understood in the art.

The term “PD-1 inhibitor” refers to a moiety (e.g., compound, nucleic acid, polypeptide, antibody) that decreases, inhibits, blocks, abrogates or interferes with the activity or expression of PD-1 (e.g., Programmed Cell Death Protein 1; PD-1 (CD279); GI: 145559515), including variants, isoforms, species homologs of human PD-1 (e.g., mouse) and analogs that have at least one common epitope with PD-1. A PD-1 inhibitor includes molecules and macromolecules such as, for example, compounds, nucleic acids, polypeptides, antibodies, peptibodies, diabodies, minibodies, single-chain variable fragments (ScFv), and fragments or variants thereof. Thus, a PD-1 inhibitor as used herein refers to any moiety that antagonizes PD-1 activity or expression. PD-1 inhibitor efficacy can be measured, for example, by its inhibitor concentration at 50% (half-maximal inhibitor concentration or IC₅₀). PD-1 inhibitors include exemplary compounds and compositions described herein. A PD-1 antibody refers to a PD-1 inhibitor which is a monoclonal or polyclonal antibody as described herein.

The terms “nivolumab,” “pembrolizumab,” “pidilizumab,” “AMP-224,” “REGN2810,” “PDR 001,”, “SHR-1210”, “SAR-439684” and “MEDI0680” are used in accordance with their plain and ordinary meaning as understood in the art.

The term “CTLA-4 inhibitor” refers to a moiety (e.g., compound, nucleic acid, polypeptide, antibody) that decreases, inhibits, blocks, abrogates or interferes with the activity or expression of CTLA-4, including variants, isoforms, species homologs of human CTLA-4 (e.g., mouse) and analogs that have at least one common epitope with CTLA-4. A CTLA-4 inhibitor includes molecules and macromolecules such as, for example, compounds, nucleic acids, polypeptides, antibodies, peptibodies, diabodies, minibodies, single-chain variable fragments (ScFv), and fragments or variants thereof. Thus, a CTLA-4 inhibitor as used herein refers to any moiety that antagonizes CTLA-4 activity or expression. CTLA-4 inhibitor efficacy can be measured, for example, by its inhibitor concentration at 50% (half-maximal inhibitor concentration or IC₅₀). CTLA-4 inhibitors include exemplary compounds and compositions described herein. A CTLA-4 antibody refers to a CTLA-4 inhibitor which is a monoclonal or polyclonal antibody as described herein.

The term “ipilimumab” is used in accordance with their plain and ordinary meaning as understood in the art.

The term “CD276 inhibitor” refers to a moiety (e.g., compound, nucleic acid, polypeptide, antibody) that decreases, inhibits, blocks, abrogates or interferes with the activity or expression of CD276 (also referred to as B7-H3), including variants, isoforms, species homologs of human CD276 (e.g., mouse) and analogs that have at least one common epitope with CD276. A CD276 inhibitor includes molecules and macromolecules such as, for example, compounds, nucleic acids, polypeptides, antibodies, peptibodies, diabodies, minibodies, single-chain variable fragments (ScFv), and fragments or variants thereof. Thus, a CD276 inhibitor as used herein refers to any moiety that antagonizes CD276 activity or expression. CD276 inhibitor efficacy can be measured, for example, by its inhibitor concentration at 50% (half-maximal inhibitor concentration or IC₅₀). CD276 inhibitors include exemplary compounds and compositions described herein. A CD276 antibody refers to a CD276 inhibitor which is a monoclonal or polyclonal antibody as described herein.

The terms “polypeptide” and “protein” are used interchangeably herein and refer to any molecule that includes at least 2 or more amino acids.

The term “Inhibitor Antibody” refers to a monoclonal or polyclonal antibody that binds to its substrate or target with sufficient strength to inhibit activity of the substrate or target. As used herein, an Inhibitor Antibody comprises a PD-L1 inhibitor antibody, PD-1 inhibitor antibody, CTLA-4 inhibitor antibody, and/or CD276 inhibitor antibody.

The term “effective amount” refers to the amount of a therapy (e.g., a combination provided herein or another active agent such as an anti-cancer agent described herein) which is sufficient to accomplish a stated purpose or otherwise achieve the effect for which it is administered. An effective amount can be sufficient to reduce and/or ameliorate the progression, development, recurrence, severity and/or duration of a given disease, disorder or condition and/or a symptom related thereto, or can be sufficient to reduce the level of activity or binding of a polypeptide (e.g., PD-L1, PD-1, CTLA-4). An effective amount can be a “therapeutically effective amount” which refers to an amount sufficient to provide a therapeutic benefit such as, for example, the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. A therapeutically effective amount of a composition described herein can enhance the therapeutic efficacy of another therapeutic agent.

The term “regimen” refers to a protocol for dosing and timing the administration of one or more therapies (e.g., combinations described herein or another active agent such as an anti-cancer agent described herein) for treating a disease, disorder, or condition described herein. A regimen can include periods of active administration and periods of rest as known in the art. Active administration periods include administration of combinations and compositions described herein and the duration of time of efficacy of such combinations and compositions. Rest periods of regimens described herein include a period of time in which no compound is actively administered, and in certain instances, includes time periods where the efficacy of such compounds can be minimal. Combination of active administration and rest in regimens described herein can increase the efficacy and/or duration of administration of the combinations and compositions described herein.

The terms “therapies” and “therapy” refer to any protocol(s), method(s), and/or agent(s) that can be used in the prevention, treatment, management, and/or amelioration of a disease, disorder, or condition or one or more symptoms thereof. In certain instances the term refers to active agents such as an anti-cancer agent described herein. The terms “therapy” and “therapy” can refer to anti-viral therapy, anti-bacterial therapy, anti-fungal therapy, anti-cancer therapy, biological therapy, supportive therapy, and/or other therapies useful in treatment, management, prevention, or amelioration of a disease, disorder, or condition or one or more symptoms thereof known to one skilled in the art, for example, a medical professional such as a physician.

The term “patient” or “subject” refers to a mammal, such as a human, bovine, rat, mouse, dog, monkey, ape, goat, sheep, cow, or deer. Generally, a patient as described herein is human.

The terms “inhibition”, “inhibit”, “inhibiting” refer to a reduction in the activity, binding, or expression of a polypeptide or reduction or amelioration of a disease, disorder, or condition or a symptom thereof. Inhibiting as used here can include partially or totally blocking stimulation, decreasing, preventing, or delaying activation or binding, or inactivating, desensitizing, or down-regulating protein or enzyme activity or binding.

Antibodies described herein can be polyclonal or monoclonal and include xenogeneic, allogeneic, or syngeneic forms and modified versions thereof (e.g., humanized or chimeric). An “antibody” is intended to mean a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa) and each amino-terminal portion of each chain includes a variable region of about 100 to about 130 or more amino acids and each carboxy-terminal portion of each chain includes a constant region (See Borrebaeck (ed.) (1995) Antibody Engineering, Second Edition, Oxford University Press.; Kuby (1997) Immunology, Third Edition, W.H. Freeman and Company, New York). Specific molecular antigens that can be bound by an antibody described herein include PD-L1, PD-1, CTLA-4, and their epitopes.

The term “monoclonal antibody(ies)” refers to a population of antibody molecules that contain one species of an antigen binding site capable of immunoreacting with a particular epitope of an antigen, whereas the term “polyclonal antibody(ies)” refers to a population of antibody molecules that contain multiple species of antigen binding sites capable of interacting with a particular antigen. A monoclonal antibody, typically displays a single binding affinity for a particular antigen with which it immunoreacts. For example, the monoclonal antibodies to be used in accordance with the present invention can be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J Mol. Biol. 222: 581-597 (1992); Sidhu et al., J. Mal. Biol. 338(2): 299-310 (2004); Lee et al., J. Mal. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lon berg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein also include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, pp. 6851-6855 (1984)). “Humanized antibody(ies)” can be considered as a subset of chimeric antibodies described herein.

The term “human” when used in reference to an antibody or a functional fragment thereof (e.g., “humanized antibody(ies))” refers an antibody or functional fragment thereof that has a human variable region or a portion thereof corresponding to human germline immunoglobulin sequences. Such human germline immunoglobulin sequences are described by Kabat et al. (1991) Sequences of Proteins of Immunological Interest, Fifth Edition, U.S. Department of Health and Human Services, NIH Publication No. 91-3242. A human antibody, in the context of the present invention, can include an antibody that binds to PD-L1 or variants thereof as described herein.

In certain instances a human antibody is an antibody that possesses an amino acid sequence corresponding to that of an antibody produced by a human and/or has been made using any of the techniques for making human antibodies as disclosed herein. Human antibodies can be produced using various techniques known in the art, including phage-display libraries. Hoogenboom and Winter, J. Mol. Biol., 227:381 (1991); Marks et al., J. Mal. Biol., 222:581 (1991). Also available for the preparation of human monoclonal antibodies are methods described in Cole et al., Monoclonal Antibodies and Cancer Therapy, Alan R. Liss, p. 77 (1985); Boemer et al., J. Immunol., 147(1):86-95 (1991). See also van Dijk and van de Winkel, Curr. Opin. Pharmacol., 0.2.: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE technology). See also, for example, Li et al., Proc. Natl. Acad. Sci. USA, 103:3557-3562 (2006) regarding human antibodies generated via a human B-cell hybridoma technology.

A “humanized antibody” refers to antibodies made by a non-human cell having variable or variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention can include amino acid residues not encoded by human germline immunoglobulin sequences (e.g., mutations introduced by random or site-specific mutagenesis in vitro or by somatic mutation in vivo), for example in the CDRs. Humanized antibodies can also include antibodies in which CDR sequences derived from the germline of another mammalian species, such as a mouse, have been grafted onto human framework sequences.

Humanized forms of non-human (e.g., murine) antibodies are antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a nonhuman species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies can comprise residues that are not found in the recipient antibody or in the donor antibody. These modifications can be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions can include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally can also include at least a portion of an immunoglobulin constant region (Fc), which can be a human immunoglobulin. Exemplary methods and humanized antibodies include those described by Jones et al. Nature 321: 522-525 (1986); Riechmann et al. Nature 332:323-329 (1988); and Presta, Curr. Op. Struct. Biol. 2:593-596 (1992); Vaswani and Hamilton, Ann. Allergy. Asthma & Immunol. 1: 105-115 (1998); Harris, Biochem. Soc. Transactions 23:1035-1038 (1995); Burle and Gross, Curr. Op. Biotech. 5:428-433 (1994); and U.S. Pat. Nos. 6,982,321 and 7,087,409.

The term “functional fragment” when used in reference to an antibody refers to a portion of the antibody including heavy or light chain polypeptides that retains some or all of the binding activity as the antibody from which the fragment was derived. Such functional fragments can include, for example, an Fd, Fv, Fab, F(ab′), F(ab)₂, F(ab′)₂, single chain Fv (ScFv), diabody, triabody, tetrabody and minibody. Other functional fragments can include, for example, heavy or light chain polypeptides, variable region polypeptides or CDR polypeptides or portions thereof so long as such functional fragments retain binding activity. Such antibody binding fragments can be found described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics, 22:189-224 (1993); Phickthun and Skerra, Meth. Enzymol., 178:497-515 (1989) and in Day, E. D., Advanced Immunochemistry, Second Ed., Wiley-Liss, Inc., New York, N.Y. (1990). Antibody Engineering, Second Edition, Oxford University Press, 1995.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while μ and ε contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domains can differ extensively in sequence between different antibodies. The variability in sequence is concentrated in the CDRs while the less variable portions in the variable domain are referred to as framework regions (FR). The CDRs of the light and heavy chains are primarily responsible for the interaction of the antibody with antigen. Numbering of amino acid positions used herein is according to the EU Index, as in Kabat et al. (1991) Sequences of proteins of immunological interest. (U.S. Department of Health and Human Services, Washington, D.C.) 5^{th} Ed. A variable region can be a human variable region.

A CDR refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. The positions of CDRs within a canonical antibody variable domain have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

For example, CDRs defined according to either the Kabat (hypervariable), Chothia (structural), or MacCallum (J. Mol. Biol. 262:732-745 (1996)) designations, as set forth in the Table 1 below:

TABLE 1
CDR Definitions

| | Kabat¹ | Chothia ² | MacCallum ³ | Loop Location |
|---|---|---|---|---|
| V_{H} CDR1 | 31-35 | 26-32 | 30-35 | linking B and C strands |
| V_{H} CDR2 | 50-65 | 53-55 | 47-58 | linking C′ and C″ strands |
| V_{H} CDR3 | 95-102 | 96-101 | 93-101 | linking F and G strands |
| V_{L} CDR1 | 24-34 | 26-32 | 30-36 | linking B and C strands |
| V_{L} CDR2 | 50-56 | 50-52 | 46-55 | linking C′ and C″ strands |
| V_{L} CDR3 | 89-97 | 91-96 | 89-96 | linking F and G strands |

¹Residue numbering follows the nomenclature of Kabat et al., supra
² Residue numbering follows the nomenclature of Chothia et al., supra

The term “cancer” refers to any physiological condition in mammals characterized by unregulated cell growth. Cancers described herein include solid tumors and hematological (blood) cancers. A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “enhance” refers to an increase or improvement in the function or activity of a protein or cell after administration or contacting with a combination described herein compared to the protein or cell prior to such administration or contact.

The term “administering” refers to the act of delivering a combination or composition described herein into a subject by such routes as oral, mucosal, topical, suppository, intravenous, parenteral, intraperitoneal, intramuscular, intralesional, intrathecal, intranasal or subcutaneous administration. Parenteral administration includes intravenous, intramuscular, intra-arteriole, intradermal, subcutaneous, intraperitoneal, intraventricular, and intracranial administration. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition).

The term “coadministration” refers to administration of two or more agents (e.g., a combination described herein and another active agent such as an anti-cancer agent described herein). The timing of coadministration depends in part of the combination and compositions administered and can include administration at the same time, just prior to, or just after the administration of one or more additional therapies, for example cancer therapies such as chemotherapy, hormonal therapy, radiotherapy, or immunotherapy. The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compounds described herein can be used in combination with one another, with other active agents known to be useful in treating cancer.

The term “anti-cancer agent” is used in accordance with its plain ordinary meaning and refers to a composition having anti-neoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” refers to a therapy or regimen that includes administration of a chemotherapeutic or anti-cancer agent described herein.

The terms “halo,” “halogen,” and “halide” refer to —F, —Cl, —Br, and —I.

The term “alkyl” by itself or as part of another substituent refers to, unless otherwise stated, a straight (e.g., unbranched) or branched carbon chain (or carbon), or combination thereof, having no unsaturation and can include mono-, di- and multivalent radicals. An alkyl as defined herein can be designated by its number of carbon atoms (e.g., C₁-C₁₀ means one to ten carbons). Alkyls herein can include C₁-C₁₀, C₁-C₈, C₁-C₆, and C₁-C₄ lengths. A “perfluoroalkyl” refers to an alkyl in which all of the hydrogens in the alkyl chain are replaced with fluoro.

The term “alkoxy” refers to an alkyl group (e.g., C₁-C₁₀, C₁-C₈, C₁-C₆, and C₁-C₄ alkyl) attached to the remainder of the molecule via an oxygen linker (—O—). Exemplary alkoxy groups include groups having the formula —OR, where R is branched or linear alkyl. A “perfluoroalkoxyl” moiety refers to an alkoxy in which all of the hydrogens in the alkyl chain are replaced with fluoro.

The term “aminoalkyl” refers to an alkyl group (e.g., C₁-C₁₀, C₁-C₈, C₁-C₆, and C₁-C₄ alkyl) in which one or more hydrogen atoms are replaced with an amino group

The term “alkylamino” refers to an alkyl group (e.g., C₁-C₁₀, C₁-C₈, C₁-C₆, and C₁-C₄ alkyl) attached to the remainder of the molecule via a nitrogen linker (—NR—). Exemplary alkylamino groups include N-methylamino, N-ethylamino, N-isopropylamino, and the like.

The term “acyl” refers to a moiety having the formula, —C(O)R, where R is a substituted or unsubstituted alkyl, haloalkyl, or amino group. The term “acylamino” refers to an acyl moiety having an attached amino group and includes, for example, such moieties as acetylamino, propionylamino, butyrylamino, isobuytrylamino, and others.

The term “alkythio” refers to an alkyl group (e.g., C₁-C₁₀, C₁-C₈, C₁-C₆, and C₁-C₄ alkyl) attached to the remainder of the molecule via a sulfur linker (—S—). Exemplary alkylthio groups include methylthio, ethylthio, propylthio, and others.

The term “heterocycle” or “heterocyclyl” refers to a stable 3- to 15-membered monocyclic group that is saturated or unsaturated and contains one or more heteroatoms (e.g., N, O, or S). Exemplary heterocycles include, but are not limited to morpholinyl, piperidinyl, piperazinyl, pyranyl, pyrrolidinyl, pyrrolinyl, imidazolidinyl, imidazolinyl, oxetanyl, azetidinyl, and others.

1. Compositions

Provided herein are combinations (e.g., combination therapies and compositions) useful for treating a variety of diseases, disorders, and symptoms thereof, including for example, cancer. The combinations described herein include an HDAC inhibitor and a PD-L1 inhibitor and/or PD-1 inhibitor, and further a CTLA-4 inhibitor. In one non-limiting example a benzamide HDAC inhibitor of formula I is provided, and examples of PD-L1 inhibitors, PD-1 inhibitors, and CTLA-4 inhibitors are described herein. In one aspect is a combination that includes a therapeutically effective amount of a PD-L1 inhibitor and/or PD-1 inhibitor, a CTLA-4 inhibitor, and a therapeutically effective amount of a compound of formula I:

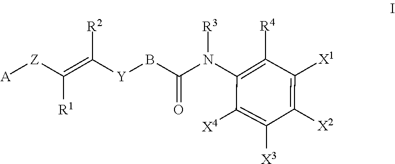

wherein:

A is a phenyl or heterocyclic group, optionally substituted with 1 to 4 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkythio, C₁-C₄ perfluoroalkyl, C₁-C₄ perfluoroalkyloxy, C₁-C₄ alkoxycarbonyl, phenyl, and a heterocyclic group;

B is phenyl optionally substituted with 1 to 3 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, C₁-C₄ alkoxycarbonyl, and phenyl;

Y is a moiety comprising —CO— which is linear and in which the distances between the centroid of ring B (W1), the centroid of ring A (W2) and an oxygen atom as a hydrogen bond acceptor in the moiety Y (W3) are: W1-W2=about 6.0 Å, W1-W3=about 3.0 Å to about 6.0 Å, and W2-W3=about 4.0 Å to about 8.0 Å, respectively;

Z is a bond or C₁-C₄ alkylene, —O—, —S—, —NH—, —CO—, —CS—, —SO—, or —SO₂—;

R¹ and R² are independently hydrogen or C₁-C₄ alkyl;

R³ is hydrogen or C₁-C₄ alkyl;

R⁴ is hydrogen or —NH₂; and

one of X¹, X², X³, or X⁴ is halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, or C₁-C₄ alkoxycarbonyl optionally substituted with halogen or C₁-C₄ alkyl, while the others of X¹, X², X³, or X⁴ are independently hydrogen,

provided that when R⁴ is hydrogen, one of X¹, X², X³, or X⁴ is —NH₂, an aminoalkyl group or an alkylamino group.

In certain instances A is phenyl or phenyl optionally substituted with halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkythio, C₁-C₄ perfluoroalkyl, C₁-C₄ perfluoroalkyloxy, C₁-C₄ alkoxycarbonyl, phenyl, or a heterocyclic group. A can be a heterocyclic group (e.g., a 5 to 10-membered heterocyclic group) containing a —N—, —S—, or —O— moiety. In certain instances A is a 5 to 10-membered N-heterocyclic moiety having 1, 2, 3, 4, or more nitrogen heteroatoms, such as for example, pyrrolidinyl, pyrrolinyl, imidazolidinyl, imdazolyl, pyrazolidinyl, pyrazolyl, oxazolidinyl, oxazolyl, thiazolidinyl, thiazolyl, piperidinyl, pyridinyl, piperizinyl, diazinyl, tetrazolyl, triazinyl, tetrazinyl, azepinyl, diazepinyl, azocanyl, or azocinyl. A can be a saturated or unsaturated 5 to 10 membered N-heterocyclic moiety. In certain instances A is a 6-membered N-heterocyclic moiety, such as for example, pyridine.

In certain embodiments, B is phenyl. B can be phenyl optionally substituted with a small moiety such as, for example, halogen, —OH, —NH₂, —NO₂, —CN, —COOH, or C₁-C₄ alkyl. In some embodiments B is phenyl substituted with halogen. In other embodiments, B is substituted with an electron donating group (EDG). In still other embodiments, B is phenyl substituted with an electron withdrawing group (EWG). In yet another embodiment, B is phenyl substituted with C₁-C₄ alkyl. B can be methyl-, ethyl-, or propyl-substituted phenyl. B can be methoxy-, ethoxy-, or propoxy-substituted phenyl.

In certain instances Y is —C(O)NH—CH₂—. In certain embodiments, Z is a bond. Z can be a methylene, ethylene, or propylene moiety. In some embodiments, Z is —O—, —S—, —NH—, —CO—, —CS—, —SO—, or —SO₂—.

R¹ and R² are in certain instances both hydrogen. R¹ and R² can both be C₁-C₄ alkyl, for example, R¹ and R² can both be methyl, ethyl, or propyl. In certain instances if one of R¹ or R² is hydrogen the other is C₁-C₄ alkyl (e.g., methyl). R³ can be hydrogen. In other embodiments, R³ is C₁-C₄ alkyl (e.g., methyl or ethyl).

R⁴ can be —NH₂. In certain instances R⁴ is —NH₂ where one of X¹, X², X³, or X⁴ is halogen. When R⁴ is —NH₂, X² or X³ can be halogen. In one embodiment R⁴ is —NH₂ and X² is halogen. In such instances X² can be —F.

In another embodiment, R′, R², and R³ are hydrogen where Z is a bond, R⁴ is —NH₂ and Y is —C(O)NH—CH₂—. In such embodiments, A can be a heterocyclic moiety as described above and B can be phenyl. X¹, X², X³, or X⁴ can be halogen (e.g., —F) or —NH₂.

The compound of formula I can be a compound as substantially described by U.S. Pat. Nos. 7,244,751 and 7,550,490 both of which are incorporated herein by reference in their entirety for all purposes. In one embodiment the compound of formula I is N-(2-amino-4-fluorophenyl)-4-[[[(2E)-1-oxo-3-(3-pyridinyl)-2-propen-1-yl]amino]methyl]benzamide. In another embodiment the compound of formula I has the formula Ia as set forth below:

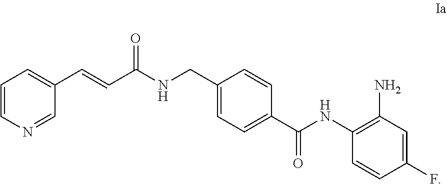 [SMILES: Nc1cc(F)ccc1NC(=O)c1ccc(CNC(=O)/C=C/c2cccnc2)cc1]

Compounds of formula I as described herein include pharmaceutically acceptable salts, pharmaceutically acceptable stereoisomers, prodrugs, enantiomers, diastereomers, hydrates, co-crystals, and polymorphs thereof.

In certain instances, the combination includes a compound of formula I (e.g., Ia) present at an amount of greater than about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. The combination can include a compound of formula I present at an amount greater than about: 1 mg, 2 mg, 3 mg, 4 mg, 5 mg, 6 mg, 7 mg, 8 mg, 9 mg, or 10 mg. In certain instances the compound of formula I is present in an amount greater than about 5 mg or about 10 mg. The combination can include a compound of formula I present at an amount greater than about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg.

The combination can include a compound present in an amount of at least about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. The combination can include a compound of formula I present at an amount of at least about: 1 mg, 2 mg, 3 mg, 4 mg, 5 mg, 6 mg, 7 mg, 8 mg, 9 mg, or 10 mg. In certain instances the compound of formula I is present in an amount of at least about 5 mg or about 10 mg. The combination can include a compound of formula I present at an amount of at least about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg.

The combination can include a compound present in an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. The combination can include a compound of formula I present at an amount of about: 1 mg, 2 mg, 3 mg, 4 mg, 5 mg, 6 mg, 7 mg, 8 mg, 9 mg, or 10 mg. In certain instances the compound of formula I is present in an amount of about 5 mg or about 10 mg. The combination can include a compound of formula I present at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg.

A compound of formula I can be present in the combinations described herein relative to the weight of the patient (e.g., mg/kg). In some instances, the compound of formula I is present in an amount equivalent to about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg. In other instances the compound of formula I is present in an amount equivalent to about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg.

### PD-L1 Inhibitors

PD-L1 inhibitors useful in the combinations described herein include any molecule capable of inhibiting, blocking, abrogating or interfering with the binding of PD-L1 to PD-1, activity or expression of PD-L1. In particular, a PD-L1 inhibitor can be a small molecule compound, a nucleic acid, a polypeptide, an antibody, a peptibody, a diabody, a minibody, a single-chain variable fragment (ScFv), or a functional fragment or variant thereof. In one instance the PD-L1 inhibitor is a small molecule compound (e.g., a compound having a molecule weight of less than about 1000 Da). In one embodiment, the PD-L1 inhibitor is CA-170 (AUPM-170; Curis, Inc.). In other instances, useful PD-L1 inhibitors in the combinations described herein include nucleic acids and polypeptides. The PD-L1 inhibitor can be a polypeptide (e.g., macrocyclic polypeptide) such as those exemplified in U.S. Patent Application Publication No.: 2014/0294898, which is incorporated herein by reference in its entirety and for all purposes. In one example, the PD-L1 inhibitor is an antibody, peptibody, diabody, minibody, ScFv, or a functional fragment thereof. In another example, the PD-L1 inhibitor is a PD-L1 inhibitor antibody. The PD-L1 inhibitor antibody can be a monoclonal or polyclonal antibody. In certain embodiments, the PD-L1 inhibitor antibody is a monoclonal antibody.

PD-L1 antibodies include all known types of antibodies and functional fragments thereof, including but not limited to, those exemplified herein such as, for example, human antibodies, mouse antibodies, chimeric antibodies, humanized antibodies, or chimeric humanized antibodies.

In one embodiment, the PD-L1 inhibitor antibody is a human antibody. In another embodiment, the PD-L1 inhibitor antibody is a mouse antibody. In still another embodiment, the PD-L1 inhibitor antibody is a chimeric antibody. In yet another embodiment, the PD-L1 inhibitor antibody is a humanized antibody. In yet another embodiment, the PD-L1 inhibitor antibody is a chimeric humanized antibody. The PD-L1 inhibitor antibody can be a human antibody or humanized antibody. The PD-L1 inhibitor antibody can be durvalumab, avelumab, atezolizumab, BMS-936559, STI-A1010, STI-A1011, STI-A1012, STI-A1013, STI-A1014, or STI-A1015. In some embodiments, two or more PD-L1 antibodies are administered in combination with a compound of formula I as described herein.

The PD-L1 inhibitor antibody can be durvalumab. Durvalumab is an Fc optimized monoclonal antibody directed against PD-L1, with potential immune checkpoint inhibitory and anti-neoplastic activities. Without being bound by any particular theory, durvalumab binds to PD-L1, thereby blocking its binding to and activation of its receptor, PD-1, which can be expressed on activated T-cells. This can reverse T-cell inactivation and activate the immune system to exert a cytotoxic T-lymphocyte (CTL) response against PD-L1-expressing tumor cells. The Fc region of durvalumab is modified in such a way that it does not induce either antibody-dependent cytotoxicity (ADCC) or complement-dependent cytotoxicity (CDC).

The PD-L1 inhibitor antibody can be avelumab. Avelumab is a human immunoglobulin G1 (IgG1) monoclonal antibody directed against PD-L1, with potential immune checkpoint inhibitory and anti-neoplastic activities. Without being bound by any particular theory, avelumab binds to PD-L1 and prevents the interaction of PD-L1 with its receptor, PD-1. This inhibits the activation of PD-1 and its downstream signaling pathways. This can restore immune function through the activation of cytotoxic T-lymphocytes (CTLs) targeted to PD-L1-overexpressing tumor cells. Avelumab appears to induce an antibody-dependent cellular cytotoxic (ADCC) response against PD-L1-expressing tumor cells.

The PD-L1 inhibitor antibody can be atezolizumab. Atezolizumab is a human, Fc optimized, monoclonal antibody directed against the protein ligand PD-L1, with potential immune checkpoint inhibitory and anti-neoplastic activities. Without being bound by any particular theory, atezolizumab binds to PD-L1, blocking its binding to and activation of its receptor, PD-1, expressed on activated T-cells, which may enhance the T-cell-mediated immune response to neoplasms and reverse T-cell inactivation. In addition, by binding to PD-L1, atezolizumab also appears to prevent binding of PD-L1 to B7.1 expressed on activated T cells, which can further enhance the T-cell-mediated immune response. The Fc region of atezolizumab is modified in such a way that it does not induce either antibody-dependent cytotoxicity (ADCC) or complement-dependent cytotoxicity (CDC).

The PD-L1 inhibitor antibody can be BMS-936559. BMS-936559 is a fully human IgG4 monoclonal antibody directed against PD-L1, with potential immune checkpoint inhibitory activity. Without being bound by any particular theory, BMS-936559 binds to PD-L1 and inhibits its binding to both PD-1 and CD80.

The PD-L1 inhibitor antibody can be STI-A1010, STI-A1011, STI-A1012, STI-A1013, STI-A1014, or STI-A1015. STI-A1010, STI-A1011, STI-A1012, STI-A1013, STI-A1014, and STI-A1015 (Sorrento Therapeutics) are fully human monoclonal antibodies that are each directed against PD-L1.

### PD-1 Inhibitors

PD-1 inhibitors useful in the combinations described herein include any molecule capable of inhibiting, blocking, abrogating or interfering with the activity or expression of PD-1. In particular, a PD-1 inhibitor can be a small molecule compound, a nucleic acid, a polypeptide, an antibody, a peptibody, a diabody, a minibody, a single-chain variable fragment (ScFv), or a functional fragment or variant thereof. In one instance the PD-1 inhibitor is a small molecule compound (e.g., a compound having a molecule weight of less than about 1000 Da.) In other instances, useful PD-1 inhibitors in the combinations described herein include nucleic acids and polypeptides. The PD-1 inhibitor can be a polypeptide (e.g., macrocyclic polypeptide) such as those exemplified in U.S. Patent Application Publication No.: 2014/0294898, which is incorporated herein by reference in its entirety and for all purposes. In one example, the PD-1 inhibitor is an antibody, peptibody, diabody, minibody, ScFv, or a functional fragment thereof. In one example, the PD-1 inhibitor is AMP-224 (GSK).

AMP-224 is a recombinant fusion protein comprising an extracellular domain of the PD-1 ligand programmed cell death ligand 2 (PD-L2) and an Fc region of human IgG. Certain cancers can evade and suppress the immune system, in part, and without being bound by any particular theory by interactions between PD-1 and B7-H1. AMP-224 appears to block this interaction and therefore appears to overcome immune suppression.

In another example, the PD-1 inhibitor is a PD-1 antibody. The PD-1 antibody can be a monoclonal or polyclonal antibody. In certain embodiments, the PD-1 antibody is a monoclonal antibody.

PD-1 antibodies include all known types of antibodies and functional fragments thereof, including but not limited to, those exemplified herein such as, for example, human antibodies, mouse antibodies, chimeric antibodies, humanized antibodies, or chimeric humanized antibodies.

In one embodiment, the PD-1 antibody is a human antibody. In another embodiment, the PD-1 antibody is a mouse antibody. In still another embodiment, the PD-1 antibody is a chimeric antibody. In yet another embodiment, the PD-1 antibody is a humanized antibody. In yet another embodiment, the PD-1 antibody is a chimeric humanized antibody. The PD-1 antibody can be a human antibody or humanized antibody. The PD-1 antibody can be nivolumab, pembrolizumab, pidilizumab, REGN2810, PDR 001, or MEDI0680. In some embodiments, two or more PD-1 antibodies are administered in combination with a compound of formula I as described herein.

The PD-1 antibody can be nivolumab. nivolumab (marketed as OPDIVO) is a fully human monoclonal antibody directed against PD-1 with immunopotentiation activity. Without being bound by any particular theory, nivolumab binds to and blocks the activation of PD-1 by its cognate ligands, resulting in the activation of T-cells and cell-mediated immune responses against tumor cells or pathogens.

The PD-1 antibody can be pembrolizumab. Pembrolizumab (MK-3475, marketed as KEYTRUDA) is a humanized monoclonal IgG4 antibody directed against human cell surface receptor PD-1 with potential immunopotentiating activity. Without being bound by any particular theory, pembrolizumab binds to PD-1, an inhibitory signaling receptor expressed on the surface of activated T cells, and blocks the binding to and activation of PD-1 by its cognate ligands. The blocking of binding and activity results in the activation of T-cell-mediated immune responses against tumor cells.

The PD-1 antibody can be pidilizumab. Pidilizumab (CT-011) is a humanized monoclonal antibody directed against human PD-1 with immunomodulating and antitumor activities. Without being bound by any particular theory, pidilizumab blocks interaction between the receptor PD-1 with its ligands, resulting in the attenuation of apoptotic processes in lymphocytes, primarily effector/memory T cells, and the augmentation of the anti-tumor activities of NK cells.

The PD-1 antibody can be REGN2810. REGN2810 is a human monoclonal antibody directed against PD-1, with potential immune checkpoint inhibitory and anti-neoplastic activity. Without being bound by any particular theory REGN2810 binds to PD-1, inhibits binding to its cognate ligand, and prevents the activation of its downstream signaling pathways. This can restore immune function through the activation of cytotoxic T-cells.

The PD-1 antibody can be PDR 001. PDR 001 is a fully humanized monoclonal antibody directed against PD-1, with immune checkpoint inhibitory and anti-neoplastic activities. Without being bound by any particular theory, PDR 001 binds to PD-1 expressed on activated T-cells and blocks the interaction with its cognate ligands. The inhibition of ligand binding prevents PD-1-mediated signaling and results in both T-cell activation and the induction of T-cell-mediated immune responses against tumor cells.

The PD-1 antibody can be MEDI0680 (AMP-514) is a monoclonal antibody directed against the PD-1, with potential immunomodulating and anti-neoplastic activity. Without being bound by any particular theory, MEDI0680 appears to inhibit the activation of PD-1 and its downstream signaling pathways. This inhibition can restore immune function through the activation both of T-cells and cell-mediated immune responses against PD-1 overexpressing tumor cells.

### CTLA-4 Inhibitors

CTLA-4 inhibitors useful in the combinations described herein include any molecule capable of inhibiting, blocking, abrogating or interfering with the activity or expression of CTLA-4. In particular, a CTLA-4 inhibitor can be a small molecule compound, a nucleic acid, a polypeptide, an antibody, a peptibody, a diabody, a minibody, a single-chain variable fragment (ScFv), or a functional fragment or variant thereof. In one instance the CTLA-4 inhibitor is a small molecule compound (e.g., a compound having a molecule weight of less than about 1000 Da.) In other instances, useful CTLA-4 inhibitors in the combinations described herein include nucleic acids and polypeptides. The CTLA-4 inhibitor can be a polypeptide (e.g., macrocyclic polypeptide). In one example, the CTLA-4 inhibitor is an antibody, peptibody, diabody, minibody, ScFv, or a functional fragment thereof. In one example, the CTLA-4 inhibitor is ipilimumab.

In another example, the CTLA-4 inhibitor is a CTLA-4 antibody. The CTLA-4 antibody can be a monoclonal or polyclonal antibody. In certain embodiments, the CTLA-4 antibody is a monoclonal antibody.

CTLA-4 antibodies include all known types of antibodies and functional fragments thereof, including but not limited to, those exemplified herein such as, for example, human antibodies, mouse antibodies, chimeric antibodies, humanized antibodies, or chimeric humanized antibodies. In one embodiment, the CTLA-4 antibody is a human antibody. In another embodiment, the CTLA-4 antibody is a mouse antibody. In still another embodiment, the CTLA-4 antibody is a chimeric antibody. In yet another embodiment, the CTLA-4 antibody is a humanized antibody. In yet another embodiment, the CTLA-4 antibody is a chimeric humanized antibody. The CTLA-4 antibody can be a human antibody or humanized antibody. The CTLA-4 antibody can be administered in combination with a compound of formula I as described herein.

### CD276 Inhibitors

CD276 (B7-H3) is a relatively newly discovered, but important member of the immune checkpoint family. CD276 is expressed on antigen-presenting cells in active/inflamed “hot” tumor micro-environments (“TMEs”) and suppresses CD8⁺ cytotoxic T cells. CD276 expression is upregulated with administration of a compound of formula I as described herein. CD276 inhibitors useful in the combinations described herein include any molecule capable of inhibiting, blocking, abrogating or interfering with the activity or expression of CD276. In particular, a CD276 inhibitor can be a small molecule compound, a nucleic acid, a polypeptide, an antibody, a peptibody, a diabody, a minibody, a single-chain variable fragment (ScFv), or a functional fragment or variant thereof. In one instance the CD276 inhibitor is a small molecule compound (e.g., a compound having a molecule weight of less than about 1000 Da.) In other instances, useful CD276 inhibitors in the combinations described herein include nucleic acids and polypeptides. The CD276 inhibitor can be a polypeptide (e.g., macrocyclic polypeptide). In one example, the CD276 inhibitor is an antibody, peptibody, diabody, minibody, ScFv, or a functional fragment thereof.

In another example, the CD276 inhibitor is a CD276 antibody. The CD276 antibody can be a monoclonal or polyclonal antibody. In certain embodiments, the CD276 antibody is a monoclonal antibody.

CD276 antibodies include all known types of antibodies and functional fragments thereof, including but not limited to, those exemplified herein such as, for example, human antibodies, mouse antibodies, chimeric antibodies, humanized antibodies, or chimeric humanized antibodies.

In one embodiment, the CD276 antibody is a human antibody. In another embodiment, the CD276 antibody is a mouse antibody. In still another embodiment, the CD276 antibody is a chimeric antibody. In yet another embodiment, the CD276 antibody is a humanized antibody. In yet another embodiment, the CD276 antibody is a chimeric humanized antibody. The CD276 antibody can be a human antibody or humanized antibody. The CD276 antibody can be administered in combination with a compound of formula I as described herein, or with any of the other compositions described herein.

A PD-L1 inhibitor antibody, PD-1 inhibitor antibody, CTLA-4 inhibitor antibody, and/or CD276 inhibitor antibody (any one of which is referred to as “Inhibitor Antibody” herein) can be of any antibody isotype. The term isotype refers to the antibody class that is encoded by heavy chain constant region genes. The heavy chains of a given antibody or functional fragment determine the class of that antibody or functional fragment: IgM, IgG, IgA, IgD or IgE. Each class can have either κ or λ. light chains. The term subclass refers to the minor differences in amino acid sequences of the heavy chains that differentiate the subclasses. In humans there are two subclasses of IgA (subclasses IgA1 and IgA2) and there are four subclasses of IgG (subclasses IgG1, IgG2, IgG3 and IgG4). Such classes and subclasses are well known to those skilled in art.

Useful Inhibitor Antibodies bind to their substrates with sufficient strength to inhibit activity of the substrate (e.g., PD-L1, PD-1, CTLA-4, and/or CD276). The term bind as used herein refers to an interaction between molecules to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions or forces. Binding of an antibody or functional fragment thereof can be detected using, for example, an enzyme-linked immunosorbant assay or any one of a number of methods that are well known to those skilled in the art.

The strength of the total non-covalent interactions between a single antigen-binding site on an Inhibitor Antibody or functional fragment and a single epitope of a target molecule is the affinity of the antibody or functional fragment for that epitope. The ratio of association (k₁) to dissociation (k₋₁) of an antibody or functional fragment thereof to a monovalent antigen (k₁/k₋₁) is the association constant K, which is a measure of affinity. The value of K varies for different complexes of antibody or functional fragment and antigen and depends on both k₁ and k₋₁. The association constant K for an antibody or functional fragment of the invention can be determined using any method provided herein or any other method well known to those skilled in the art.

The affinity at one binding site does not always reflect the true strength of the interaction between an antibody or functional fragment and an antigen. When complex antigens containing multiple, repeating antigenic determinants come in contact with antibodies containing multiple binding sites, the interaction of such an antibody or functional fragment with antigen at one site will increase the probability of a reaction at a second site. The strength of such multiple interactions between a multivalent antibody and antigen is called the avidity. The avidity of an antibody or functional fragment can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

The specificity of an Inhibitor Antibody or functional fragment thereof refers to the ability of an individual antibody or functional fragment thereof to react with only one antigen (e.g., a single epitope of PD-L1, PD-1, and CTLA-4). An antibody or functional fragment can be considered specific when it can distinguish differences in the primary, secondary or tertiary structure of an antigen or isomeric forms of an antigen.

The Inhibitor Antibody can be present in an amount as a measure with regards to the weight of the patient in need thereof. For example, the Inhibitor Antibody can be present in an amount of about: 0.1 mg/kg to about 50 mg/kg, 0.1 mg/kg to about 40 mg/kg, 0.1 mg/kg to about 30 mg/kg, 0.1 mg/kg to about 25 mg/kg, 0.1 mg/kg to about 20 mg/kg, 0.1 mg/kg to about 15 mg/kg, 0.1 mg/kg to about 10 mg/kg, 0.1 mg/kg to about 7.5 mg/kg, 0.1 mg/kg to about 5 mg/kg, 0.1 mg/kg to about 2.5 mg/kg, or about 0.1 mg/kg to about 1 mg/kg. The Inhibitor Antibody can be present in an amount of about: 0.5 mg/kg to about 50 mg/kg, 0.5 mg/kg to about 40 mg/kg, 0.5 mg/kg to about 30 mg/kg, 0.5 mg/kg to about 25 mg/kg, 0.5 mg/kg to about 20 mg/kg, 0.5 mg/kg to about 15 mg/kg, 0.5 mg/kg to about 10 mg/kg, 0.5 mg/kg to about 7.5 mg/kg, 0.5 mg/kg to about 5 mg/kg, 0.5 mg/kg to about 2.5 mg/kg, or about 0.5 mg/kg to about 1 mg/kg. The Inhibitor Antibody can be present in an amount of about 0.5 mg/kg to about 5 mg/kg or about 0.1 mg/kg to about 10 mg/kg. The Inhibitor Antibody can be present in an amount of about 0.1 mg/kg to about 20 mg/kg or about 0.1 mg/kg to about 30 mg/kg.

In still other embodiments, the Inhibitor Antibody can be present at an amount of about: 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, or 50 mg/kg. The Inhibitor Antibody can be present at an amount of about: 1 mg/kg, 2 mg/kg, 3 mg/kg, 5 mg/kg, 10 mg/kg, 15 mg/kg, 20 mg/kg, 25 mg/kg, or 30 mg/kg. The Inhibitor Antibody can be present at an amount of about: 3 mg/kg, 10 mg/kg, 20 mg/kg, or 30 mg/kg.

The Inhibitor Antibody can be present in the combination at an amount of about: 1 mg, 5 mg, 10 mg, 15 mg, 20 mg, 25 mg, 30 mg, 40 mg, 50 mg, 60 mg, 70 mg, 75 mg, 80 mg, 90 mg, 100 mg, 150 mg, or 200 mg. The Inhibitor Antibody can be present in the combination at an amount of about: 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg. The Inhibitor Antibody can be present in the combination at an amount of about 1000 mg to about 2000 mg. The Inhibitor Antibody can be present in the combination at an amount of about: 1 mg to about 10 mg, 10 mg to about 20 mg, 25 mg to about 50 mg, 30 mg to about 60 mg, 40 mg to about 50 mg, 50 mg to about 100 mg, 75 mg to about 150 mg, 100 mg to about 200 mg, 200 mg to about 500 mg, 500 mg to about 1000 mg, 1000 mg to about 1200 mg, 1000 mg to about 1500 mg, 1200 mg to about 1500 mg, or 1500 to about 2000 mg.

The Inhibitor Antibody can be present in the combination in an amount of about 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 40 mg/mL, 50 mg/mL, 60 mg/mL, 70 mg/mL, 80 mg/mL, 90 mg/mL, 100 mg/mL, 150 mg/mL, 200 mg/mL, 250 mg/mL, 300 mg/mL, 400 mg/mL, or 500 mg/mL. In one embodiment, the Inhibitor Antibody is present in the combination in an amount of about: 1 mg/mL to about 10 mg/mL, 5 mg/mL to about 10 mg/mL, 5 mg/mL to about 15 mg/mL, 10 mg/mL to about 25 mg/mL; 20 mg/mL to about 30 mg/mL; 25 mg/mL to about 50 mg/mL, or 50 mg/mL to about 100 mg/mL.

In certain instances the therapeutically effective amount of an Inhibitor Antibody is determined as an amount provided in a package insert provided with the Inhibitor Antibody. The term package insert refers to instructions customarily included in commercial packages of medicaments approved by the FDA or a similar regulatory agency of a country other than the USA, which contains information about, for example, the usage, dosage, administration, contraindications, and/or warnings concerning the use of such medicaments.

Compounds of formula I as described herein can be provided in amounts that are synergistic with the amount of the PD-L1 and/or PD-1 inhibitor, and a CTLA-4 inhibitor. The term synergistic refers to a combination described herein (e.g., a compound of formula I and a PD-L1 and/or PD-1 inhibitor, plus a CTLA-4 inhibitor—including coadministration with another active agent such as an anti-cancer agent described herein) or a combination of regimens such as those described herein that is more effective than the additive effects of each individual therapy or regimen.

A synergistic effect of a combination described herein can permit the use of lower dosages of one or more of the components of the combination (e.g., a compound of formula I, or a PD-L1 inhibitor, or a PD-1 inhibitor, or a CTLA-4 inhibitor). A synergistic effect can permit less frequent administration of at least one of the administered therapies (e.g., a compound of formula I, or a PD-L1 inhibitor, or a PD-1 inhibitor, or a CTLA-4 inhibitor) to a subject with a disease, disorder, or condition described herein. Such lower dosages and reduced frequency of administration can reduce the toxicity associated with the administration of at least one of the therapies (e.g., a compound of formula I, or a PD-L1 inhibitor, or a PD-1 inhibitor, or a CTLA-4 inhibitor) to a subject without reducing the efficacy of the treatment. A synergistic effect as described herein avoid or reduce adverse or unwanted side effects associated with the use of any therapy.

2. Pharmaceutical Compositions

Combinations described herein can be provided as a pharmaceutical composition suitable for administration via any route to a patient described herein including but not limited to: oral, mucosal (e.g., nasal, inhalation, pulmonary, sublingual, vaginal, buccal, or rectal), parenteral (e.g., subcutaneous, intravenous, bolus injection, intramuscular, or intra-arterial), topical (e.g., eye drops or other ophthalmic preparations), transdermal or transcutaneous administration to a patient.

Exemplary of dosage forms include: tablets; caplets; capsules (e.g., gelatin capsules); cachets; lozenges; suppositories; powders; gels; liquid dosage forms suitable for parenteral administration to a patient; and sterile solids (e.g., crystalline or amorphous solids) that can be reconstituted to provide liquid dosage forms suitable for parenteral administration to a patient.

Pharmaceutical compositions and dosage forms described herein typically include one or more excipients. Suitable excipients are well known to those skilled in the art of pharmacy. Whether a particular excipient is suitable for incorporation into a pharmaceutical composition or dosage form depends on a variety of factors such as, for example, the intended route of administration to the patient. Pharmaceutical compositions described herein can include other agents such as stabilizers, lubricants, buffers, and disintegrants that can reduce the rate by which an active ingredient can decompose in a particular formulation.

Pharmaceutical compositions described herein can in certain instances include additional active agents other than those in the combinations described herein (e.g., an anti-cancer agent such as those described herein) in an amount provided herein.

In one embodiment, the compound of formula I is provided in an oral dosage form such as a tablet or capsule. In another embodiment, the compound of formula I is supplied as a powder (e.g., lyophilized powder) that can be resuspended in a liquid suitable for parenteral administration.

PD-L1 inhibitors, PD-1 inhibitors, and CTLA-4 inhibitors described herein can be provided in forms convenient to or facilitate their administration to a patient. For example, where the inhibitor is an Inhibitor Antibody as described herein, the inhibitor can be formulated as a ready to use solution for parenteral administration. In other examples, the inhibitor, including for example an Inhibitor Antibody, can be formulated as a powder (e.g., lyophilized powder) that can be resuspended in a liquid suitable for parenteral administration. In one embodiment, the combination includes an Inhibitor Antibody formulated for intravenous administration. In still another embodiment the combination includes a compound of formula I formulated as an oral dosage form (e.g., a tablet or capsule) and an Inhibitor Antibody formulated for intravenous administration.

Combinations described herein can be provided as controlled release pharmaceutical products, which have a goal of improving drug therapy over that achieved by their non-controlled counterparts. Controlled release formulations can extend activity of the drug, reduce dosage frequency, and increase subject compliance. In addition, controlled release formulations can be used to affect the time of onset of action or other characteristics, such as blood levels of the drug, and can thus affect the occurrence of side (e.g., adverse) effects.

3. Kits

The combinations and pharmaceutical compositions described herein can be provided as part of a kit. Such kits can, for example, improve patient compliance or improve the accuracy or ease of preparation for administering the combination. The kit includes a compound of formula I where the compound is supplied in a formulation as described herein.

Kits of the invention can include the combinations described herein having the same or different formulation. Each component of a combination described herein in a kit can be supplied in a separate, individual container. Alternatively or additionally, components of the combinations described herein can be supplied in a single container. In such instances, the container can be a container that is ready for administration to a patient in need thereof, such as for example, an IV bag, ampoule, or a syringe. In one embodiment, the compound of formula I in the kit is formulated for oral administration (e.g., a tablet, capsule, or sachet).

The contents of kits described herein can be provided in sterile form. The kit and its contents can be provided in a form that is ready for administration to the subject in need. In such instances, the components of the combination of the kit are supplied as a formulation and optionally in an administration device such that administration requires little to no further action by the user. Where kits include administration devices, such devices include devices known and understood by those skilled in the art for routes of administration described herein, such as but not limited to, syringes, pumps, bags, cups, inhalers, droppers, patches, creams, or injectors.

4. Method

The combinations, pharmaceutical compositions, and kits described herein are useful for treating diseases, disorders, or alleviating or eliminating the symptoms of diseases and disorders such as, for example, cancer. It is to be understood that the methods described herein pertain to administration of combinations and pharmaceutical compositions described herein, and such combinations and pharmaceutical compositions can be provided in the form of a kit as described herein. Provided herein are methods of treating cancer by administering a therapeutically effective amount of a combination described herein to a patient in need thereof. Also provided herein are methods of managing cancer by administering therapeutically effective amount of a combination described herein to a patient in need thereof.

In some embodiments, the combination is used to treat cancer. In some embodiments, the cancer is a cancer described herein.

In some embodiments, the combination is an HDAC inhibitor (HDACi) a PD-L1 inhibitor, and a CTLA-4 inhibitor. In some embodiments, the combination is an HDAC inhibitor (HDACi) a PD-1 inhibitor, and a CTLA-4 inhibitor.

Combinations useful in the methods described herein include a compound of formula I:

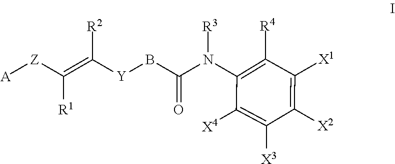

where:

A is a phenyl or heterocyclic group, optionally substituted with 1 to 4 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkythio, C₁-C₄ perfluoroalkyl, C₁-C₄ perfluoroalkyloxy, C₁-C₄ alkoxycarbonyl, phenyl, and a heterocyclic group;

B is phenyl optionally substituted with 1 to 3 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, C₁-C₄ alkoxycarbonyl, and phenyl;

Y is a moiety comprising —CO— which is linear and in which the distances between the centroid of ring B (W1), the centroid of ring A (W2) and an oxygen atom as a hydrogen bond acceptor in the moiety Y (W3) are: W1-W2=about 6.0 Å, W1-W3=about 3.0 Å to about 6.0 Å, and W2-W3=about 4.0 Å to about 8.0 Å, respectively;

Z is a bond or C₁-C₄ alkylene, —O—, —S—, —NH—, —CO—, —CS—, —SO—, or —SO₂—;

R¹ and R² are independently hydrogen or C₁-C₄ alkyl;

R³ is hydrogen or C₁-C₄ alkyl;

R⁴ is hydrogen or —NH₂; and

one of X¹, X², X³, or X⁴ is halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, or C₁-C₄ alkoxycarbonyl optionally substituted with halogen or C₁-C₄ alkyl, while the others of X¹, X², X³, or X⁴ are independently hydrogen, provided that when R⁴ is hydrogen, one of X¹, X², X³, or X⁴ is —NH₂, an aminoalkyl group or an alkylamino group.

Compounds of formula I useful in the methods described herein include compounds as substantially described hereinabove. In certain instances, the compound of formula I used to treat cancer in the methods provided herein includes compounds where R¹, R², and R³ are hydrogen. In certain instances Y is —C(O)NH—CH₂—. In certain instances, R³ can be C₁-C₄ alkyl as described above. A of formula I can be a 5 to 10-membered heterocyclic moiety. In particular, and as described above, useful embodiments of the compound of formula I include compounds where A is N-heterocycle, such as for example, a 5 or 6 membered heterocyclic moiety. A can be, in certain instances, a pyridinyl.

The compound of formula I useful in the methods described herein can be a compound where R⁴ is —NH₂ amount of 0.1 mg/kg, 0.3 mg/kg, 1 mg/kg, 2 mg/kg, 3 mg/kg, 5 mg/kg, 10 mg/kg, or 20 mg/kg and at least of X¹, X², X³, or X⁴ is —NH₂ or halogen. In certain instances, the compound of formula I for use in the methods described herein includes compounds where R⁴ is —NH₂ and at least one of X¹, X², X³, or X⁴ is halogen (e.g., —F). In one embodiment, the compound of formula I is a compound having the structure of formula Ia as set forth above.

The PD-L1 inhibitors, PD-1 inhibitors, and CTLA-4 inhibitors for use in the methods described herein are those inhibitors described herein. For example, the PD-L1 inhibitors, PD-1 inhibitors, and CTLA-4 inhibitors can be a small molecule compound, a nucleic acid, a polypeptide, an antibody, a peptibody, a diabody, a minibody, a single-chain variable fragment (ScFv), or functional fragment or variant thereof. In other examples, the inhibitor can be an Inhibitor Antibody as set forth above.

### Target Cancers

The cancer can be a solid tumor. The cancer can be a hematological cancer. In certain instances, the cancer is a solid tumor selected from the group consisting of squamous cell carcinoma, non-squamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, colorectal cancer, pancreatic cancer, lymphoma, leiomyosarcoma, liposarcoma, synovial sarcoma, or malignant peripheral sheath tumor (MPNST).

In particular embodiments, the cancer is a solid tumor selected from non-small cell lung cancer (NSCLC), hepatocellular carcinoma, melanoma, ovarian cancer, breast cancer, pancreatic cancer, renal cell carcinoma, or colorectal cancer. The cancer can be non-small cell lung cancer (NSCLC). The cancer can be hepatocellular carcinoma. The cancer can be melanoma. The cancer can be ovarian cancer. The cancer can be breast cancer. The cancer can be pancreatic cancer. The cancer can be renal cell carcinoma. The cancer can be colorectal cancer.

Provided herein are methods of treating NSCLC by administering a therapeutically effective amount of a combination described herein where the combination includes a compound of formula I and an Inhibitor Antibody. In some embodiments, the NSCLC is Stage IIA or Stage IIB. The NSCLC can be a Stage IIIA or Stage IIIB cancer. The NSCLC can be a Stage IV cancer. Staging of cancers as described herein is described by the American Joint Committee on Cancer TNM classification of malignant tumors cancer staging notation as is well understood in the art. Those of skill in the art will readily understand other staging classification systems are available and applicable to the methods described herein. In certain instances, the method is a method of treating Stage IIIA or IIIB NSCLC by administering a combination described herein that includes a compound of formula I and an Inhibitor Antibody.

Still further provided herein are methods of treating melanoma by administering a therapeutically effective amount of a combination described herein where the combination includes a compound of formula I and an Inhibitor Antibody. In some embodiments the melanoma is a Stage IIA, IIB, or IIC cancer. In another embodiment, the melanoma is a Stage IIIA, Stage IIIB, or Stage IIIC cancer. In still another embodiment, the melanoma is a Stage IV cancer. In one aspect the method is a method of treating Stage II (e.g., Stage IIA, IIB, or IIC) melanoma by administering a therapeutically effective amount of a combination described herein where the combination includes a compound of formula I and an Inhibitor Antibody.

Also provided herein are methods of treating breast cancer by administering a therapeutically effective amount of a combination described herein where the combination includes a compound of formula I and an Inhibitor Antibody. The breast cancer can be HER2 negative breast cancer. The breast cancer can be a HER2 positive breast cancer. The breast cancer can be triple-negative breast cancer. In some embodiments the breast cancer is a Stage IA or Stage D3 cancer. In another embodiment, the breast cancer is a Stage IIA or Stage IIB cancer. In still another embodiment, the breast cancer is a Stage IIIA, Stage IIIB, or Stage IIIC cancer. In yet another embodiment, the breast cancer is a Stage IV cancer.

In other embodiments, the cancer is a hematological cancer selected from lymphoma, Non-Hodgkin's lymphoma (NHL), Hodgkin's Lymphoma, Reed-Sternberg disease, multiple myeloma (MM), acute myelogenous leukemia (AML), chronic myelogenous leukemia (CIVIL), acute lymphocytic leukemia, (ALL), or chronic lymphocytic leukemia (CLL). In certain embodiments, the cancer is Hodgkin's Lymphoma or Reed-Sternberg disease.

The combinations described herein can be administered to a cancer patient at any time following diagnosis. For example, the cancer patient can be treatment naive (e.g., has not received a cancer therapy for the diagnosed cancer). The cancer patient can be treatment naive for one cancer but can be diagnosed with one or more other cancers resulting from, for example, metastasis or malignancy. The cancer patient can be immune checkpoint naive for one or more cancers. The cancer patient can have a cancer that is refractory. In certain instances, the combinations described herein are administered as a first line therapy (e.g., the first therapy administered to a treatment naive cancer patient) to a patient in need thereof

However, cancer morbidity and mortality are often associated with ineffective therapy or a cancer gaining resistant to or becoming refractory to one or more cancer therapies. The combinations described herein can, therefore, be administered to patients in need thereof as a second, third, fourth, fifth, sixth, or more line of treatment. The combinations described herein can be administered to a cancer patient who has been treated with at least one anti-cancer therapy or anti-cancer agent. In certain instances the patient has received at least one anti-cancer therapy including, for example, chemotherapy, radiotherapy, surgery, targeted therapy, immunotherapy, or a combination thereof. The patient can have a cancer that is resistant/refractory to treatment with at least one anti-cancer agent.

The methods of treating cancers herein include treating subjects who have been treated with a checkpoint inhibitor and have experienced no response to treatment, or a partial response, or stable disease, but then develop resistance to treatment with progression of disease or who have experienced a complete response to treatment, but then develop resistance to treatment with progression of disease (as defined by RECIST or other criteria). Resistance is defined as disease progression during treatment or a lack of response to treatment. Such Inhibitor Antibody treatment failures can be treated with an Inhibitor Antibody in combination with an HDAC inhibitor, such as, without limitation, HBI-8000 or an HDAC inhibitor that inhibits cancer-associated Class I HDAC selected from one or more of HDAC1, HDAC2, or HDAC3. In some instances the HDAC inhibitor also inhibits Class IIb HDAC1.

Response Criteria

RECIST:

RECIST is a set of established criteria or standards, internationally recognized for evaluating patient response, stability and progression in clinical trials and in the clinical practice. Originally published in 2000, and revised in 2009 (Eisenhauer E A, et al.; New response criteria in solid tumors: revised RECIST guideline (version 1.1); Eur J Cancer 2009; 45:228-47), as a joint effort of the European Organization for Research and Treatment of Cancer, the National Cancer Institute of the United States and the National Cancer Institute of Canada Clinical Trials Group, RECIST has traditionally been utilized in the evaluation of response to chemotherapy.

Evaluation of Target Lesions:

Complete Response (CR): Disappearance of all target lesions; Partial Response (PR): At least a 30% decrease in the sum of the LD (longest diameter) of target lesions, taking as reference the baseline sum LD; Stable Disease (SD): Neither sufficient shrinkage to qualify for PR nor sufficient increase to qualify for PD, taking as reference the smallest sum LD since the treatment started; Progressive Disease (PD): At least a 20% increase in the sum of the LD of target lesions, taking as reference the smallest sum LD recorded since the treatment started or the appearance of one or more new lesions.

Evaluation of Non-Target Lesions

Complete Response (CR): Disappearance of all non-target lesions and normalization of tumor marker level; Incomplete Response/Stable Disease (SD): Persistence of one or more non-target lesion(s) or/and maintenance of tumor marker level above the normal limits; Progressive Disease (PD): Appearance of one or more new lesions and/or unequivocal progression of existing non-target lesions.

Other Response Criteria

Other response criteria include the Immune-Related Response Criteria or iRECIST, as defined by Wolchok et al., in 2009 (Wolchok J D, et al.; Guidelines for the Evaluation of Immune Therapy Activity in Solid Tumors: Immune-Related Response Criteria. Clin Cancer Res 2009; 15(23):7412-20) and the revised International Working Group Response Criteria (Cheson B D et al., Revised response criteria for malignant lymphoma. J. Clin. Oncol. 2007; 25:579-586).

The methods of treating cancer include methods for inhibiting cell growth by administering a therapeutically effective amount of a combination described herein where the combination includes a compound of formula I and a PD-L1 inhibitor and/or PD-1 inhibitor, plus a CTLA-4 inhibitor described herein.

Also provided herein are methods of inhibiting metastasis of a cancer in a patient in need thereby by administering a therapeutically effective amount of a combination described herein where the combination includes a compound of formula I and a PD-L1 inhibitor and/or PD-1 inhibitor, plus a CTLA-4 inhibitor described herein. In some embodiments, metastasis is inhibited by at least about 5%, 10%, 20%, 30%, 40%, 50%, 60%, 70%, 80%, 90%, or 100%.

In another aspect is a method of reducing pre-existing tumor metastasis in a cancer patient in need thereof by administering a therapeutically effective amount of a combination described herein where the combination includes a compound of formula I and a PD-L1 inhibitor and/or PD-1 inhibitor, plus a CTLA-4 inhibitor described herein. In some embodiments, pre-existing tumor metastasis is reduced by at least about 5%, 10%, 20%, 30%, 40%, 50%, 60%, 70%, 80%, 90%, or 100%.

In still another aspect the methods of treating cancer also provide for methods for reducing tumor burden in an individual by administering a therapeutically effective amount of a combination described herein where the combination includes a compound of formula I and a PD-L1 inhibitor and/or PD-1 inhibitor, plus a CTLA-4 inhibitor described herein. In some embodiments, tumor burden is reduced by at least about 5%, 10%, 20%, 30%, 40%, 50%, 60%, 70%, 80%, 90%, or 100%.

In another aspect the methods of treating cancer also provide for methods for reducing tumor burden in an individual by administering a therapeutically effective amount of a combination described herein where the combination includes a compound of formula I and a PD-L1 inhibitor and/or PD-1 inhibitor, plus a CTLA-4 inhibitor described herein. In some embodiments, tumor burden is reduced by at least about 5%, 10%, 20%, 30%, 40%, 50%, 60%, 70%, 80%, 90%, or 100%.

The methods of treating cancer described herein also provide for methods for increasing or otherwise prolonging time to disease progression of certain stages (including advanced stages of cancer such as Stage III and IV cancer described herein). Time to disease progression can be prolonged in a patient by administering a therapeutically effective amount of a combination described herein where the combination includes a compound of formula I and a PD-L1 inhibitor and/or PD-1 inhibitor, plus a CTLA-4 inhibitor described herein. In some embodiments, the increase is a comparison between the time to disease progression without treatment and with treatment with a combination described herein. In some embodiments, the methods described herein prolong the time to disease progression by at least 1 week, 2 weeks, 3 weeks, 4 weeks, 1 month, 2 months, 3 months, 4 months, 5 months, 6 months, 7 months, 8 months, 9 months, 10 months, 11 months, 1 year, or more, including values therein.

The methods of treating cancer described herein also provide for methods for increasing or otherwise prolonging survival (including overall survival) of patients diagnosed with cancer as described herein. Patient survival can be prolonged by administering a therapeutically effective amount of a combination described herein where the combination includes a compound of formula I and a PD-L1 inhibitor and/or PD-1 inhibitor, plus a CTLA-4 inhibitor described herein. In some embodiments, the increase is a comparison between the survival without treatment and with treatment with a combination as described herein. In some embodiments, the methods described herein prolong survival by at least 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, or more, including values therein.

The methods of treating cancer described herein also provide for methods for increasing progression-free survival of patients diagnosed with cancer as described herein. Patient progression-free survival can be prolonged by administering a therapeutically effective amount of a combination described herein where the combination includes a compound of formula I and a PD-L1 inhibitor and/or PD-1 inhibitor, plus a CTLA-4 inhibitor described herein. In some embodiments, the increase is a comparison between the progression-free survival without treatment and with treatment with a combination as described herein. In some embodiments, the methods described herein increase progression-free survival by at least 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, or more, including values therein.

Also provided herein are methods of reducing a level of myeloid-derived suppressor cells (MDSC) in a patient in need thereof by administering an effective amount of a combination described herein where the combination includes a compound of formula I and a PD-L1 inhibitor and/or PD-1 inhibitor, plus a CTLA-4 inhibitor described herein. The reduction of MDSC can benefit the treatment of a cancer described herein. The level of MDSC in a human patient can be measured before, during, and after administration of a combination described herein. In some embodiments, it can be useful to compare pre- and post-administration amounts of MDSC in the patient. A reduction in the amount, level, or number of MDSC following administration can indicate effectiveness of the combination in, for example, treating a cancer described herein. MD SC levels can be monitored over the course of a treatment or regimen described herein with a combination described herein. In such instances, the determination of MD SC levels at various points during the course of administration can indicate the effectiveness of the regimen.

Methods of reducing the percentage or level of Treg cells in a patient in need thereof are also provided herein. Such methods include administering an effective amount of a combination described herein where the combination includes a compound of formula I and a PD-L1 inhibitor and/or PD-1 inhibitor, plus a CTLA-4 inhibitor described herein. The reduction of Treg cells can benefit the treatment of a cancer described herein. The level of Treg cells in a human patient can be measured before, during, and after administration of a combination described herein. In some embodiments, it can be useful to compare pre- and post-administration amounts of Treg cells in the patient. A reduction in the amount, level, or number of Treg cells following administration can indicate effectiveness of the combination in, for example, treating a cancer described herein. Treg cell levels can be monitored over the course of a treatment or regimen described herein with a combination described herein. In such instances, the determination of Treg cell levels at various points during the course of administration can indicate the effectiveness of the regimen.

The combinations described herein can be useful in methods of enhancing activity of natural killer (NK) cells. The combinations described herein can also be useful in methods of enhancing activity of cytotoxic T-cells. The methods of enhancing include contacting a NK cell or cytotoxic T-cell with a combination described herein where the combination enhances the activity of the NK cell or cytotoxic T-cell relative to its activity prior to the contact. In some embodiments, the enhanced activity of the NK cell or cytotoxic T-cell is in a cancer patient who has been administered a combination as described herein.

The combinations described herein can also enhance antibody-dependent cell-mediated cytotoxicity in a cancer patient upon administration of a combination as described herein.

The combinations described herein can include administration of each therapy (e.g., a compound of formula I and a PD-L1 inhibitor and/or PD-1 inhibitor, plus a CTLA-4 inhibitor), where the administration is performed simultaneously or sequentially (in either order). In one embodiment, the compound of formula I and the PD-L1 inhibitor and/or PD-1 inhibitor, plus a CTLA-4 inhibitor are administered simultaneously (e.g., within at least 1 to 5 min of each other). In another embodiment, the compound of formula I and the PD-L1 inhibitor and/or PD-1 inhibitor, plus a CTLA-4 inhibitor are administered sequentially (e.g., within at least 10 min, 15 min, 30 min, 1 h, 2 h, 5 h, 10 h, 12 h, 1 day, 2 days, 5 days, 7 days, 14 days, or 21 days of each other).

The compound of formula I can be administered, for example, once a day (QD), twice daily (BID), once a week (QW), twice weekly (BIW), three times a week (TIW), or monthly (QM) regularly on a continuous base or intermittent base such as BIW for 3 months then resume a month later. For example, the compound of formula I can be administered BID. The compound of formula I can be administered TIW. In certain instances, the compound of formula I is administered 2 to 3 times a week. In another embodiment, the compound of formula I is administered QD. The compound can be administered QD for about: 1 day to about 7 days, 1 day to about 14 days, 1 day to about 21 days, 1 day to about 28 days, or daily until disease progression or unacceptable toxicity. The administration of a compound of formula I can, in part, depend upon the tolerance of the patient where greater tolerance can allow greater or more frequent administration. Alternatively, where a patient shows poor tolerance to a compound of formula I, a less amount of the compound or a less frequent dosing can be performed. Compounds of formula I can be administered in any regimen as described herein.

For example, a compound of formula I can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QD. For example, a compound of formula I can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, BIW. For example, a compound of formula I can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, TIW. For example, a compound of formula I can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, QW. For example, a compound of formula I can be administered at an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg, Q2W. For example, a compound of formula I can be administered at an amount of about 5 mg or about 10 mg, QD. For example, a compound of formula I can be administered at an amount of about 5 mg or about 10 mg, BIW. For example, a compound of formula I can be administered at an amount of about 5 mg or about 10 mg, TIW. For example, a compound of formula I can be administered at an amount of about 5 mg or about 10 mg, QW. For example, a compound of formula I can be administered at an amount of about 5 mg or about 10 mg, Q2W. Administration of a compound of formula I can be continuous. Administration of a compound of formula I can be intermittent.

For example, a compound of formula I can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, QD. For example, a compound of formula I can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, BIW. For example, a compound of formula I can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, TIW. For example, a compound of formula I can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, QW. For example, a compound of formula I can be administered at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg, Q2W. Administration of a compound of formula I can be continuous. Administration of a compound of formula I can be intermittent.

r example, a compound of formula I can be administered at an amount of about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QD. For example, a compound of formula I can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, BIW. For example, a compound of formula I can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, TIW. For example, a compound of formula I can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, QW. For example, a compound of formula I can be administered at an amount of about: 0.0001 mg/kg to about 200 mg/kg, 0.001 mg/kg to about 200 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg, Q2W. In one example, a compound of formula I can be administered at an amount of about 15 mg/kg to about 75 mg/kg, QD. In another example, a compound of formula I can be administered at an amount of about 20 mg/kg to about 50 mg/kg. In still another example, a compound of formula I can be administered at an amount of about 0.001 mg/kg, 0.01 mg/kg, 0.05 mg/kg, 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 40 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. Administration of a compound of formula I can be continuous. Administration of a compound of formula I can be intermittent.

For example, a compound of formula I can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, QD. For example, a compound of formula I can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, BIW. For example, a compound of formula I can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, TIW. For example, a compound of formula I can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, QW. For example, a compound of formula I can be administered at an amount of about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg, Q2W. In one example, a compound of formula I can be administered at an amount of about 15 mg/kg to about 75 mg/kg, QD. In another example, a compound of formula I can be administered at an amount of about 20 mg/kg to about 50 mg/kg. In still another example, a compound of formula I can be administered at an amount of about 0.001 mg/kg, 0.01 mg/kg, 0.05 mg/kg, 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, 30 mg/kg, 40 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. Administration of a compound of formula I can be continuous. Administration of a compound of formula I can be intermittent.

As used herein, the term daily is intended to mean that a therapeutic compound of a combination described herein, such as a compound of formula I, is administered once or more than once each day for a period of time. The term continuous is intended to mean that a therapeutic compound of a combination described herein, such as a compound of formula I, is administered daily for an uninterrupted period of at least 10 days to 52 weeks. The term intermittent or intermittently as used herein is intended to mean stopping and starting at either regular or irregular intervals. For example, intermittent administration of a therapeutic compound of a combination described herein, such as a compound of formula I, includes administration for one to six days per week (e.g., 2 to 3 times per week or QD), administration in cycles (e.g., daily administration for two to eight consecutive weeks, then a rest period with no administration at least one day), or, for example, administration on alternate days.

Where the inhibitor is an Inhibitor Antibody, it can be administered according to established regimens such as those provided in a package insert. The Inhibitor Antibody can be administered in an amount described herein and can be administered QW, once every 2 weeks (Q2W), once every 3 weeks (Q3W), or once every 4 weeks (Q4W). In one embodiment, the Inhibitor Antibody is administered Q2W or Q4W. In another embodiment, the Inhibitor Antibody is administered Q2W. In yet another embodiment, the Inhibitor Antibody is administered Q3W. In still another embodiment, the Inhibitor Antibody is administered BIW for at least 3 weeks. In still another embodiment, the Inhibitor Antibody is administered Q4W.

For example, the Inhibitor Antibody can be administered at an amount of about 0.1 mg/kg to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 10 mg/kg, 12 mg/kg, 15 mg/kg, 20 mg/kg, 25 mg/kg, 30 mg/kg), QW. For example, the Inhibitor Antibody can be administered at an amount of about 0.1 mg/kg to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 10 mg/kg, 12 mg/kg, 15 mg/kg, 20 mg/kg, 25 mg/kg, 30 mg/kg), Q2W. For example, the Inhibitor Antibody can be administered at an amount of about 0.1 mg/kg to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 10 mg/kg, 12 mg/kg, 15 mg/kg, 20 mg/kg, 25 mg/kg, 30 mg/kg), Q4W. For example, the Inhibitor Antibody can be administered at an amount of about 0.1 mg/kg to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 10 mg/kg, 12 mg/kg, 15 mg/kg, 20 mg/kg, 25 mg/kg, 30 mg/kg), B4W (twice every 4 weeks). For example, the Inhibitor Antibody can be administered at an amount of about 0.1 mg/kg to about 30 mg/kg (including for example 0.1 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 10 mg/kg, 12 mg/kg, 15 mg/kg, 20 mg/kg, 25 mg/kg, 30 mg/kg), Q3W. For example, the Inhibitor Antibody can be administered at an amount of about 1000 mg to about 2000 mg (including for example 1000 mg, 1100 mg, 1200 mg, 1300 mg, 1400 mg, 1500 mg, 1600 mg), Q2W. For example, the Inhibitor Antibody can be administered at an amount of about 1000 mg to about 2000 mg (including for example 1000 mg, 1100 mg, 1200 mg, 1300 mg, 1400 mg, 1500 mg, 1600 mg), Q3W. For example, the Inhibitor Antibody can be administered at an amount of about 1000 mg to about 2000 mg (including for example 1000 mg, 1100 mg, 1200 mg, 1300 mg, 1400 mg, 1500 mg, 1600 mg), Q4W. Administration of the Inhibitor Antibody can be continuous. Administration of the Inhibitor Antibody can be intermittent.

The Inhibitor Antibody can be administered as an intravenous infusion over about 10, 20, 30, 40, 50, or 60 or more minutes. the Inhibitor Antibody can be administered as an intravenous infusion over about 60 minutes once every 1, 2, 3, 4, 5 or more weeks. the Inhibitor Antibody can be administered as an intravenous infusion over about 60 minutes once every two weeks. the Inhibitor Antibody can be administered as an intravenous infusion over about 60 minutes once every three weeks. the Inhibitor Antibody can be administered as an intravenous infusion over about 60 minutes once every four weeks. the Inhibitor Antibody can be administered as an intravenous infusion according to a package insert. Administration of Inhibitor Antibody can be continuous. Administration of Inhibitor Antibody can be intermittent.

The combinations described herein can be administered in a regimen. The regimen can be structured to provide therapeutically effective amounts of a compound of formula I and an inhibitor, such as an Inhibitor Antibody, over a predetermined period of time (e.g., an administration time). The regimen can be structured to limit or prevent side-effects or undesired complications of each of the components of the combination described herein. The regimen can be structured in a manner that results in increased effect for both therapies of the combination (e.g., synergy). Regimens useful for treating cancer can include any number of days of administration which can be repeated as necessary. Administration periods can be broken by a rest period that includes no administration of at least one therapy. For example, a regimen can include administration periods that include 2, 3, 5, 7, 10, 15, 21, 28, or more days. These periods can be repeated. For example, a regimen can include a set number of days as previously described where the regimen is repeated 1, 2, 3, 4, 5, 6, 7, 8, 9, 10, 11, 12, 13, or more times.

Regimens can include a rest period of at least 1, 2, 3, 5, 7, 10, or more days, where at least one therapy is no longer administered to a patient. The rest period can be determined by, for example, monitoring the reaction of the patient to the drug or by measuring the efficacy of the treatment. A rest period can be applicable to a single therapy, such that only one therapy of a combination described herein is discontinued in the rest period but the other therapy(ies) are still administered. Rest periods can be applied to all of the therapies administered to the subject such that the subject receives no therapy for a set period of time during the rest period.

Regimens described herein for the treatment of cancer using the combinations described herein can be continued until disease progression or unacceptable toxicity.

Regimens for administration of combinations described herein include, for example administration of a compound of formula I BIW or TIW and administration of a PD-L1 and/or PD-1 inhibitor, plus CTLA-4 inhibitor. For example, a compound of formula I can be administered QD for about 21 days and an Inhibitor Antibody described herein can be administered Q2W or Q4W). For example, a compound of formula I can be administered BIW or TIW and an Inhibitor Antibody described herein can be administered Q2W. In another exemplary regimen, a compound of formula I can be administered BIW or TIW and an Inhibitor Antibody can be administered BIW for 2 or 3 weeks. In still another exemplary regimen, a compound of formula I can be administered BIW or TIW and an Inhibitor Antibody can be administered Q4W. In still another exemplary regimen, a compound of formula I can be administered BIW and an inhibitor described herein can be administered Q2W, Q3W, or Q4W. In certain instances, such regimens include administration of an Inhibitor Antibody administered Q2W, Q3W, or Q4W. In yet another exemplary regimen, a compound of formula I can be administered TIW and an inhibitor described herein can be administered Q2W, Q3W, or Q4W. In certain instances, such regimens include administration of an Inhibitor Antibody administered Q2W, Q3W, or Q4W. In certain instances, such regimens include administration of a compound of formula I administered QD. In certain instances, such regimens include administration of a compound of formula I administered QD for at least 21 days. In yet another exemplary regimen, a compound of formula I can be administered QD or QW and an inhibitor (e.g., an Inhibitor Antibody) is administered Q2W, Q3W, or Q4W.

The regimen can be a regimen for administration of an Inhibitor Antibody with a compound of formula I as described herein. In one exemplary regimen including an Inhibitor Antibody, a compound of formula I can be administered BIW or TIW and an Inhibitor Antibody is administered in accordance with the prescribing information provided in, for example, a package insert. In another exemplary regimen, an Inhibitor Antibody is administered at an amount of about 1 mg/kg to about 20 mg/kg on day 1 of the regimen, and Q2W thereafter until disease progression or unacceptable toxicity and a compound of formula I is administered BIW or TIW over the same period of time. In another exemplary regimen, an Inhibitor Antibody is administered at an amount of about 1 mg/kg to about 20 mg/kg on day 1 of a regimen, and Q3W thereafter until disease progression or unacceptable toxicity and a compound of formula I is administered BIW or TIW over the same period of time. an Inhibitor Antibody can be administered Q4W with a compound of formula I, where the compound of formula I is administered, for example, BIW or TIW during the course of such a regimen. an Inhibitor Antibody can be administered Q2W with a compound of formula I, where the compound of formula I is administered, for example, BIW or TIW during the course of such a regimen. In still another exemplary regimen, an Inhibitor Antibody can be administered Q2W or Q4W with a compound of formula I, where the compound of formula I is administered, for example, QD or QW during the course of such a regimen. Such regimens can be repeated as described above (e.g., 1, 2, 3, 4, 5, 6, 7, 8, 9, 10, 11, 12, or more times).

In another exemplary regimen including an Inhibitor Antibody, a compound of formula I can be administered QD and an Inhibitor Antibody is administered in accordance with the prescribing information provided in, for example, a package insert. In another exemplary regimen, an Inhibitor Antibody is administered at an amount of about 1 mg/kg to about 20 mg/kg on day 1 of the regimen, and Q2W thereafter until disease progression or unacceptable toxicity and a compound of formula I is administered QD over the same period of time. In another exemplary regimen, an Inhibitor Antibody is administered at an amount of about 1 mg/kg to about 20 mg/kg on day 1 of a regimen, and Q3W thereafter until disease progression or unacceptable toxicity and a compound of formula I is administered QD over the same period of time. an Inhibitor Antibody can be administered Q4W with a compound of formula I, where the compound of formula I is administered QD during the course of such a regimen. an Inhibitor Antibody can be administered Q2W with a compound of formula I, where the compound of formula I is administered QD during the course of such a regimen. Such regimens can be repeated as described above (e.g., 1, 2, 3, 4, 5, 6, 7, 8, 9, 10, 11, 12, or more times).

It should also be appreciated that the combinations described herein for treating cancer can be coadministered with other active agents other than those present in the combinations described herein (e.g., anti-cancer agents). Regimens for administration of a combination described herein, including the exemplary regimens set forth above, can be modified as necessary to include administration of such active agents. Administration of such active agents, e.g., anti-cancer agents, can be performed QD, QW, QM, BID, BIW, TIW, Q2W, Q3W, or Q4W, or in accordance with prescribing information for such anti-cancer agents as set forth, for example, in a package insert. Exemplary anti-cancer agents include but are not limited to: ABRAXANE; abiraterone; ace-11; aclarubicin; acivicin; acodazole hydrochloride; acronine; actinomycin; acylfulvene; adecypenol; adozelesin; adriamycin; aldesleukin; all trans-retinoic acid (ATRA); altretamine; ambamustine; ambomycin; ametantrone acetate; amidox; amifostine; aminoglutethimide; aminolevulinic acid; amrubicin; amsacrine; anagrelide; anastrozole; andrographolide; antarelix; anthramycin; aphidicolin glycinate; apurinic acid; ara-CDP-DL-PTBA; arginine deaminase; ARRY-162; ARRY-300; ARRY-142266; AS703026; asparaginase; asperlin; asulacrine; atamestane; atrimustine; axinastatin 1; axinastatin 2; axinastatin 3; azasetron; azatoxin; azatyrosine; azacitidine; AZD8330; azetepa; azotomycin; balanol; batimastat; BAY 11-7082; BAY 43-9006; BAY 869766; bendamustine; benzochlorins; benzodepa; benzoylstaurosporine; beta-alethine; betaclamycin B; betulinic acid; b-FGF inhibitor; bicalutamide; bisantrene; bisaziridinylspermine; bisnafide; bisnafide dimesylate; bistratene A; bisantrene hydrochloride; bleomycin; bleomycin sulfate; busulfan; bizelesin; breflate; bortezomib; brequinar sodium; bropirimine; budotitane; buthionine sulfoximine; bryostatin; cactinomycin; calusterone; calcipotriol; calphostin C; camptothecin derivatives; capecitabine; carboxamide-amino-triazole; carboxyamidotriazole; CaRest M3; CARN 700; caracemide; carbetimer; carboplatin; carmustine; carubicin hydrochloride; carzelesin; castanospermine; cecropin B; cedefingol; celecoxib; cetrorelix; chlorins; chloroquinoxaline sulfonamide; cicaprost; chlorambucil; Chlorofusin; cirolemycin; cisplatin; CI-1040; cis-porphyrin; cladribine; clomifene analogues; clotrimazole; collismycin A; collismycin B; combretastatin A4; combretastatin analogue; conagenin; crambescidin 816; crisnatol; crisnatol mesylate; cryptophycin 8; cryptophycin A derivatives; curacin A; cyclopentanthraquinones; cycloplatam; cypemycin; cyclophosphamide; cytarabine; cytarabine ocfosfate; cytolytic factor; cytostatin; dacarbazine; dactinomycin; daunorubicin; daunorubicin hydrochloride; decarbazine; dacliximab; dasatinib; decitabine; dehydrodidemnin B; deslorelin; dexamethasone; dexifosfamide; dexrazoxane; dexverapamil; dexormaplatin; dezaguanine; dezaguanine mesylate; diaziquone; didemnin B; didox; diethylnorspermine; dihydro 5 azacytidine; dihydrotaxol; 9-dioxamycin; diphenyl spiromustine; docosanol; dolasetron; docetaxel; doxorubicin; doxorubicin hydrochloride; doxifluridine; droloxifene; droloxifene citrate; dromostanolone propionate; dronabinol; duazomycin; duocarmycin SA; ebselen; ecomustine; edelfosine; edrecolomab; edatrexate; eflornithine hydrochloride; eflornithine; elemene; emitefur; elsamitrucin; enloplatin; enpromate; epipropidine; epirubicin; epirubicin hydrochloride; epristeride; erbulozole; eribulin; esorubicin hydrochloride; estramustine; estramustine phosphate sodium; etanidazole; etoposide; etoposide phosphate; etoprine; exemestane; fadrozole; fadrozole hydrochloride; fazarabine; fenretinide; filgrastim; finasteride; flavopiridol; flezelastine; fluasterone; floxuridine; fludarabine phosphate; fludarabine; fluorodaunorubicin hydrochloride; forfenimex; formestane; fluorouracil; floxouridine; flurocitabine; fosquidone; fostriecin sodium; fostriecin; fotemustine; gadolinium texaphyrin; gallium nitrate; galocitabine; ganirelix; gelatinase inhibitors; gemcitabine; geldanamycin; gossyphol; GDC-0973; GSK1120212/trametinib; herceptin; hydroxyurea; hepsulfam; heregulin; hexamethylene bisacetamide; hypericin; ibandronic acid; ibrutinib; idarubicin; idarubicin hydrochloride; ifosfamide; canfosfamide; ilmofosine; iproplatin; idoxifene; idramantone; ilmofosine; ilomastat; imidazoacridones; imatinib (e.g., GLEEVEC); imiquimod; iobenguane; iododoxorubicin; ipomeanol; irinotecan; irinotecan hydrochloride; irsogladine; isobengazole; isohomohalicondrin B; itasetron; iimofosine; interleukin Il (including recombinant interleukin IL-2; or r1L.sub.2); interferon alfa-2a; interferon alfa-2b; interferon alfa-nl; interferon alfa-n3; interferon beta-la; interferon gamma-lb; jasplakinolide; kahalalide F; lamellarin N triacetate; lanreotide; leinamycin; lenograstim; lentinan sulfate; leptolstatin; letrozole; leuprorelin; levamisole; liarozole; lissoclinamide 7; lobaplatin; lombricine; lometrexol; lonidamine; losoxantrone; lovastatin; loxoribine; lurtotecan; lutetium texaphyrin; lysofylline; lanreotide acetate; lapatinib; letrozole; leucovorin; leuprolide acetate; liarozole hydrochloride; lometrexol sodium; lomustine; lenalidomide; lenvatinib; losoxantrone hydrochloride; LY294002; pomalidomide; maitansine; mannostatin A; marimastat; masoprocol; maspin; matrilysin inhibitors; menogaril; merbarone; meterelin; methioninase; metoclopramide; MIF inhibitor; mifepristone; miltefosine; mirimostim; mitoguazone; mitolactol; mitonafide; mitoxantrone; mofarotene; molgramostim; mopidamol; mycaperoxide B; myriaporone; maytansine; mechlorethamine hydrochloride; megestrol acetate; melengestrol acetate; melphalan; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nafarelin; nagrestip; napavin; naphterpin; nartograstim; nedaplatin; nemorubicin; neridronic acid; nilutamide; nisamycin; nitric oxide modulators; nitroxide antioxidant; nitrullyn; nocodazole; nogalamycin; oblimersen (GENASENSE); octreotide; okicenone; oligonucleotides; onapristone; ondansetron; ondansetron; oracin; oral cytokine inducer; ormaplatin; oxisuran; oxaloplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; porfiromycin; prednisone; prostaglandin J2; pyrazoloacridine; paclitaxel; PD035901; PD184352; PD318026; PD98059; peliomycin; pentamustine; peplomycin sulfate; PKC412; pipobroman; piposulfan; piroxantrone hydrochloride; plicamycin; plomestane; podophyllotoxin; polyphenol E; porfimer sodium; porfiromycin; prednimustine; procarbazine; procarbazine hydrochloride; puromycin; puromycin hydrochloride; pyrazofurin; raltitrexed; ramosetron; retelliptine demethylated; rhizoxin; rituximab; RII retinamide; rogletimide; rohitukine; romurtide; roquinimex; rubiginone B 1; ruboxyl; riboprine; romidepsin; safingol; safingol hydrochloride; saintopin; sarcophytol A; sargramostim; semustine; sizofiran; sobuzoxane; sodium borocaptate; sodium phenylacetate; solverol; sonermin; sorafenib; sunitinib; sparfosic acid; spicamycin D; spiromustine; splenopentin; spongistatin 1; Spongistatin 2; Spongistatin 3; Spongistatin 4; Spongistatin 5; Spongistatin 6; Spongistatin 7; Spongistatin 8; and Spongistatin 9; squalamine; stipiamide; stromelysin inhibitors; sulfinosine; suradista; suramin; swainsonine; SB239063; selumetinib/AZD6244; simtrazene; SP600125; sparfosate sodium; sparsomycin; spirogermanium hydrochloride; spiroplatin; streptonigrin; streptozocin; sulofenur; tallimustine; tamoxifen methiodide; tauromustine; tazarotene; tecogalan sodium; tegafur; tellurapyrylium; temoporfin; temozolomide; teniposide; tetrachlorodecaoxide; tetrazomine; thaliblastine; thiocoraline; thrombopoietin; thymalfasin; thymopoietin receptor agonist; thymotrinan; tirapazamine; titanocene bichloride; topsentin; toremifene; tretinoin; triacetyluridine; triciribine; trimetrexate; triptorelin; tropisetron; turosteride; tyrphostins; talisomycin; TAK-733; taxotere; tegafur; teloxantrone hydrochloride; teroxirone; testolactone; thiamiprine; thioguanine; thiotepa; tiazofurin; tirapazamine; toremifene citrate; trastuzumab; trestolone acetate; triciribine phosphate; trimetrexate; trimetrexate glucuronate; triptorelin; tubulozole hydrochloride; tumor necrosis factor-related apoptosis-inducing ligand (TRAIL); UBC inhibitors; ubenimex; U0126; uracil mustard; uredepa; vapreotide; variolin B; velaresol; veramine; verteporfin; vinorelbine; vinxaltine; vitaxin; vinblastine; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; wortmannin; XL518; zanoterone; zeniplatin; zilascorb; zinostatin stimalamer; zinostatin; and zorubicin hydrochloride.

Other exemplary anti-cancer agents include Erbulozole (e.g., R-55104); Dolastatin 10 (e.g., DLS-10 and NSC-376128); Mivobulin isethionate (e.g., CI-980); NSC-639829; Discodermolide (e.g., NVP-XX-A-296); ABT-751 (Abbott; e.g., E-7010); Altorhyrtin A; Altorhyrtin C); Cemadotin hydrochloride (e.g., LU-103793 and NSC-D-669356); Epothilone A; Epothilone B; Epothilone C; Epothilone D; Epothilone E; Epothilone F; Epothilone B N-oxide; Epothilone A N-oxide; 16-aza-epothilone B; 21-aminoepothilone B; 21-hydroxyepothilone D; 26-fluoroepothilone; Auristatin PE (e.g., NSC-654663); Soblidotin (e.g., TZT-1027); LS-4559-P (Pharmacia; e.g., LS-4577); LS-4578 (Pharmacia; e.g., LS-477-P); LS-4477 (Pharmacia); LS-4559 (Pharmacia); RPR-112378 (Aventis); DZ-3358 (Daiichi); FR-182877 (Fujisawa; e.g., WS-9265B); GS-164 (Takeda); GS-198 (Takeda); KAR-2 (Hungarian Academy of Sciences); B SF-223651 (BASF; e.g., ILX-651 and LU-223651); SAH-49960 (Lilly/Novartis); SDZ-268970 (Lilly/Novartis); AM-97 (Armad/Kyowa Hakko); AM-132 (Armad); AM-138 (Armad/Kyowa Hakko); IDN-5005 (Indena); Cryptophycin 52 (e.g., LY-355703); AC-7739 (Ajinomoto; e.g., AVE-8063A and CS-39.HCl); AC-7700 (Ajinomoto; e.g., AVE-8062; AVE-8062A; CS-39-L-Ser.HCl; and RPR-258062A); Vitilevuamide; Tubulysin A; Canadensol; CA-170 (Curis, Inc.); Centaureidin (e.g., NSC-106969); T-138067 (Tularik; e.g., T-67; TL-138067 and TI-138067); COBRA-1 (Parker Hughes Institute; e.g., DDE-261 and WHI-261); H10 (Kansas State University); H16 (Kansas State University); Oncocidin A1 (e.g., BTO-956 and DIME); DDE-313 (Parker Hughes Institute); Fijianolide B; Laulimalide; SPA-2 (Parker Hughes Institute); SPA-1 (Parker Hughes Institute; e.g., SPIKET-P); 3-IAABU (Cytoskeleton/Mt. Sinai School of Medicine; e.g., MF-569); Narcosine (e.g., NSC-5366); Nascapine; D-24851 (Asta Medica); A-105972 (Abbott); Hemiasterlin; 3-BAABU (Cytoskeleton/Mt. Sinai School of Medicine; e.g., MF-191); TMPN (Arizona State University); Vanadocene acetylacetonate; T-138026 (Tularik); Monsatrol; lnanocine (e.g., NSC-698666); 3-IAABE (Cytoskeleton/Mt. Sinai School of Medicine); A-204197 (Abbott); T-607 (Tuiarik; e.g., T-900607); RPR-115781 (Aventis); Eleutherobins (e.g., Desmethyleleutherobin; Desaetyleleutherobin; lsoeleutherobin A; and Z-Eleutherobin); Caribaeoside; Caribaeolin; Halichondrin B; D-64131 (Asta Medica); D-68144 (Asta Medica); Diazonamide A; A-293620 (Abbott); NPI-2350 (Nereus); Taccalonolide A; TUB-245 (Aventis); A-259754 (Abbott); Diozostatin; (−)-Phenylahistin (e.g., NSCL-96F037); D-62638 (Asta Medica); D-62636 (Asta Medica); Myoseverin B; D-43411 (Zentaris; e.g., D-81862); A-289099 (Abbott); A-318315 (Abbott); HTI-286 (e.g., SPA-110; trifluoroacetate salt) (Wyeth); D-82317 (Zentaris); D-82318 (Zentaris); SC-12983 (NCI); Resverastatin phosphate sodium; BPR-OY-007 (National Health Research Institutes); and SSR-250411 (Sanofi)); goserelin; leuprolide; triptolide; homoharringtonine; topotecan; itraconazole; deoxyadenosine; sertraline; pitavastatin; clofazimine; 5-nonyloxytryptamine; vemurafenib; dabrafenib; gefitinib (IRESSA); erlotinib (TARCEVA); cetuximab (ERBITUX); lapatinib (TYKERB); panitumumab (VECTIBIX); vandetanib (CAPRELSA); afatinib/BIBW2992; CI-1033/canertinib; neratinib/HKI-272; CP-724714; TAK-285; AST-1306; ARRY334543; ARRY-380; AG-1478; dacomitinib/PF299804; OSI-420/desmethyl erlotinib; AZD8931; AEE726; pelitinib/EKB-569; CUDC-101; WZ8040; WZ4002; WZ3146; AG-490; XL647; PD153035; 5-azathioprine; 5-aza-2′-deoxycytidine; 17-N-Allylamino-17-Demethoxygeldanamycin (17-AAG); 20-epi-1,25 dihydroxyvitamin D3; 5 ethynyluracil; and BMS-599626.

In certain embodiments, the combinations described herein are coadministered with an anti-cancer agent described above, where the anti-cancer agent has known activity against a particular cancer (e.g., gemcitibine coadministered with a combination described herein for treating pancreatic cancer). The anti-cancer agents above can be approved for use in treating certain indications (e.g., certain cancers) at concentrations, amounts, and using treatment regimens known in the art.

It is understood that modifications which do not substantially affect the activity of the various embodiments of this invention are also included within the definition of the invention provided herein.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

### EXAMPLES

The following examples are included for illustrative purposes only and are not intended to limit the scope of the invention.

### Example 1

The rationale for pursuing cancer immunotherapy as a therapeutic option has been driven by a long history of evidence that tumors can be recognized as non-self (similar to immune detection of pathogens like virus-infected cells), rather than as self (normal tissue). The immune system sees tumors as non-self mostly through the by early detection of molecules displayed on tumor cells that can be recognized as foreign by the immune system, which ideally becomes activated and effectively attacks and eliminates the tumor cells. A number of steps must precede an activated immune response, during which antitumor immune cells can enter (infiltrate) and engage malignant cells within the now immunologically inflamed or “hot” tumor. This is commonly referred to as the Cancer-Immunity Cycle (Chen et al). Tumor cells however can adapt over time and evade or become resistant to an antitumor immune response. A number of such resistance mechanisms are now known, and the currently approved immunotherapies have been developed to block these some of these resistance mechanisms. For example, the antibodies directed against the CTLA-4 ligand (Yervoy®, ipilumumab), the PD-1 receptor (Opdivo®, nivolumab; Keytruda®, pembrolizumab, and others) or its ligand PD-L1 (Tecentriq®, atezolizumab; and others) target these immune checkpoints and, at least in a proportion of patients, relieve antitumor resistance mediated through the CTLA-4/B7.1 and B7.2 immune checkpoint inhibitory axis or PD-1/PD-L1 checkpoint inhibitory axis. Although the use of these immune checkpoint targeting antibodies has resulted in significantly improved patient benefit and produced remarkable clinical responses in various cancers, a significant number of patients have tumors that are either inherently resistant or develop resistance, their tumors become non-inflamed, lack immune cell infiltrates (TILs) and are referred to as immunologically “cold” and the disease eventually progresses. For this reason, rational drug combinations with these checkpoint inhibitors and other immunomodulating agents are imperative to improve the rate of durable responses and patient survival. (See West A C and Johnstone R W. (2014) New and emerging HDAC inhibitors for cancer treatment. J Clin Invest 124 (1): 30-39).

HBI-8000 is a histone deacetylase inhibitor (HDACi), which as an epigenetic regulator that can change the expression of genes, up or down, without changing the DNA sequence, and therefore has the ability to alter the expression of genes which are aberrant, silenced or overexpressed in cancer cells [West and Johnstone, 2014]. Recently there have been several reports describing the ability of some HDACi (e.g., HBI-8000) to enhance antitumor immunity through positive effects on a number of the resistance mechanisms tumor acquire and can restore an inflamed or “hot” immune tumor environment with immune cell infiltrates that become active and drive antitumor immunity.

The immunomodulatory effects of HBI-8000 on the tumor micro-environment (“TME”) have been well documented. HBI-8000 administration increases the influx of CD8⁺ T cells and NK cells, and improves their function. HBI-8000 administration also reduces the number and activity of regulatory T cells (TREGs), myeloid-derived suppressor cells (MDSCs), and promotes conversion of M2 (suppressive) to M1 (antitumor) macrophages. HBI-8000 administration also increases PD-1 and PD-L1 expression in “cold” tumors, along with several other important immune signatures indicative of cold to hot conversion—this process starts early and increases over time, as does the number of responders. HBI-8000 drives positive changes in dendritic cell scores and signatures in the TME, positive changes in antigen presentation, processing, and display pathways, e.g. MHC Class I and Class II expression (mechanisms of tumor evasion). HBI-8000 increases the ratio of active CD8⁺ effector T cells to “exhausted” CD8 T cells and the cytotoxic score and signature, implying re-activation of inactive tumor selective T cells. HBI-8000 drives changes in the tumor cells themselves, priming and sensitizing them to the antitumor immune response—increasing apoptosis scores and signatures, indicating re-expression of the apoptotic machinery needed for killing the tumor cells. HBI-8000 driven TME changes also result in increased presence and activity of NK cells and M1 macrophages (innate immune system), both of which contribute to the overall antitumor immune response.

To determine the efficacy of HBI-8000 in combination with anti-CTLA-4 and anti-PD-1 checkpoint inhibitor antibodies, a study was conducted using the syngeneic MC38 colon adenocarcinoma in female C57BL/6 mice. The present study consisted of eleven groups (n=9 or 8 of female C57BL/6 mice bearing subcutaneous MC38 tumors (mean volume: 106 mm³-111 mm³) on Day 1 of the study, when dosing was initiated. Vehicle (10% Hydroxypropyl-(3-Cyclodextrin, 10% Propylene glycol in DI water, pH 2.5) and HBI-8000 (50 mg/kg) were administered orally (p.o.), once daily for twenty one days (qd×21). Anti-CTLA-4 was administered intraperitoneally (i.p.) at a dose of 2.5 mg/kg on days 1, 4, and 7. Anti-PD-1 was administered i.p. at 5 mg/kg, twice a week for two weeks (biw×2).

TABLE 2

| | Number | | | |
|---|---|---|---|---|
| Group | of | | | |
| No. | animals | Treatment 1 | Treatment 2 | Treatment 3 |
| 1 | 8 | Vehicle | | |
| 2 | 9 | HBI-8000 | | |
| | | 50 mg/kg; qd × 21 | | |
| 3 | 8 | CTLA-4 mAb | | |
| | | 2.5 mg/kg; d1, d4, d7 | | |
| 4 | 8 | PD-1 mAb | | |
| | | 5 mg/kg; biw × 2 | | |
| 5 | 8 | CTLA-4 mAb | HBI-8000 | |
| | | 2.5 mg/kg; d1, d4, d7 | 50 mg/kg; | |
| | | | qd × 21 | |
| 6 | 9 | CTLA-4 mAb | PD-1 mAb | |
| | | 2.5 mg/kg; d1, d4, d7 | 5 mg/kg; | |
| | | | biw × 2 | |
| 7 | 8 | CTLA-4 mAb | PD-1 mAb | HBI-8000 |
| | | 2.5 mg/kg; d1, d4, d7 | 5 mg/kg; | 50 mg/kg; |
| | | | biw × 2 | qd × 21 |

CTLA-4 mAb = hybridoma clone 9H10
PD-1 mAb = hybridoma clone RMPI-14

Animals were euthanized when tumor volumes reached 3000 mm³ or on the last day of the study (Day 43), whichever came first, and the time to endpoint (TTE) was calculated. Treatment outcome was determined from percent tumor growth delay (% TGD), defined as the percent increase in median TTE for treated versus control mice, with differences between the treatment groups deemed statistically significant at P<0.05 using logrank survival analysis. Mice were also monitored for complete regression (CR) and partial regression (PR) responses. Treatment tolerability was assessed by body weight (BW) measurements and frequent observation for clinical signs of treatment-related (TR) side effects.

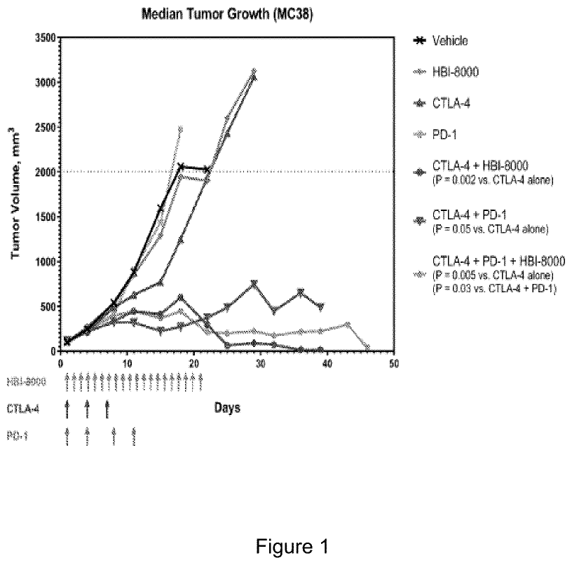

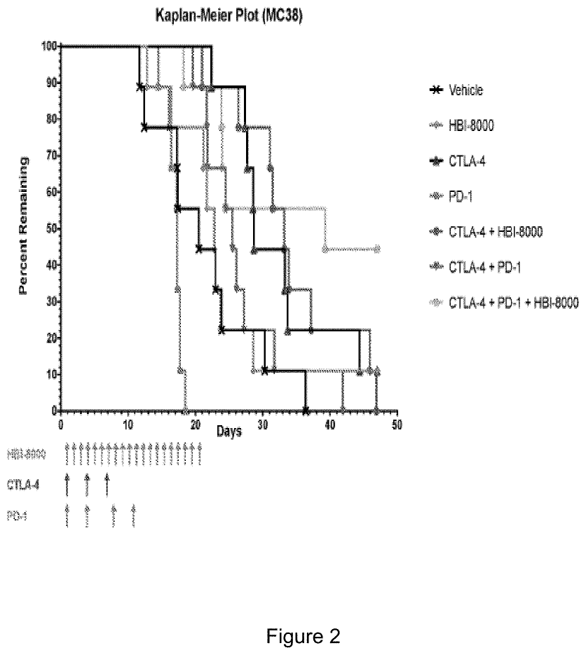

The results of these experiments are found in FIG. 1. The triplet combination therapy comprising a compound of formula I, a CTLA-4 inhibitor as described herein, and a PD-1 inhibitor as described herein resulted in statically significant tumor volume reduction. The subjects in the triplet combination group also showed a 40-50% survival rate at the conclusion of the experiments. (See FIG. 2). Collectively, these results show that the compounds of formula I enhance the activity of the triplet combination therapy.

### Example 2

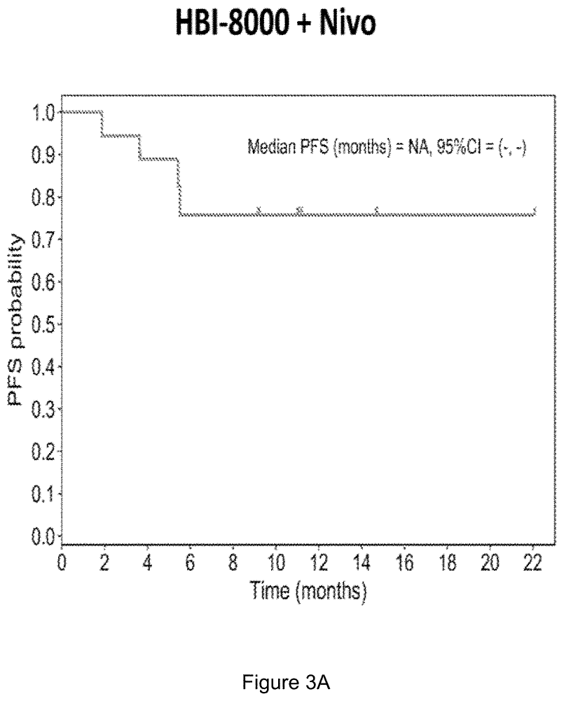

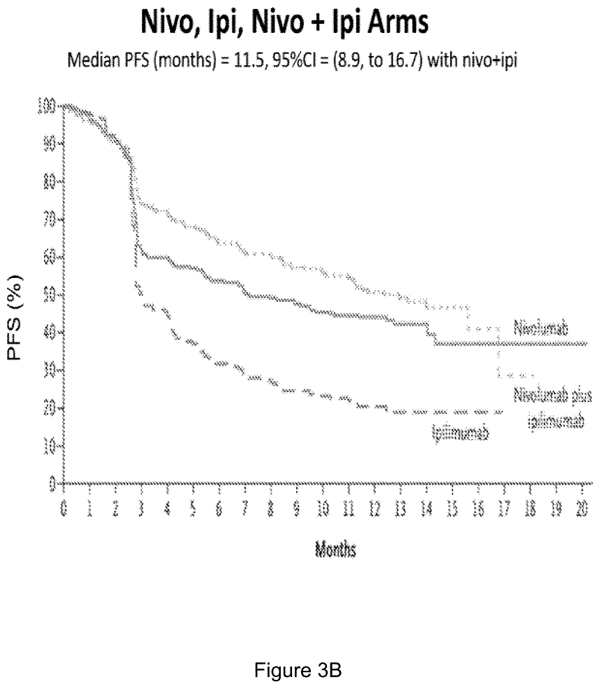

FIG. 3A depicts the probability of progression free survival (“PFS”) in terms of months resulting from a combination therapy comprising compounds of formula I and nivolumab in melanoma. This probability was generated from the results published in the New England Journal of Medicine showing the PFS for patients treated with nivolumab monotherapy, ipilimumab monotherapy, or a nivolumab plus ipilimumab combination therapy. (See FIG. 3B). The “tailing” of the plot in FIG. 3B from the combination therapy suggests a synergistic effect between the nivolumab and ipilimumab.

To test this hypothesis, compounds of formula I were administered in combination with nivolumab to 20 patients suffering from melanoma (“MEL”) (15 of these patients represented 1^{st} line of treatment), 11 patients suffering from renal cell carcinoma (“RCC”), and 13 patients suffering from non-small cell lung cancer (“NSCLC”). Prior to testing, the safety profiles of various dosages of the compounds of formula I were tested by administering escalating doses of the compounds of formula I in combination with the standard dose of nivolumab. A 30 mg BIW was established. The profile of the 20 MEL patients is depicted in Table 3.

TABLE 3
Checkpoint-Naïve Melanoma Patients (N = 20)

| Characteristics | | |
|---|---|---|
| Male/Female, n (%) | 13/7 | (65/35) |
| Median age, years (range) | 64.5 | (28-79) |
| <65, n (%) | 10 | (50) |
| 66-75 | 6 | (30) |
| >75 | 4 | (20) |
| ECOG Score, n (%) | | |
| 0 | 13 | (65) |
| 1 | 7 | (35) |
| Stage at study entry, n (%) | | |
| M1a | 5 | (25) |
| M1b | 12 | (60) |
| M1c | 3 | (15) |
| Elevated LDH, n (%) | 2 | (10) |
| Median tumor burden (target lesions), mm (range) | 38 | (10-167) |
| Median time since diagnosis, months (range) | 13.8 | (0.7-66.1) |
| BRAF status, n (%) | | |
| Mutated | 4 | (20) |
| Wildtype | 1 | (5) |
| Unknown | 15 | (75) |
| Prior surgery, n (%) | 17 | (85) |
| Prior radiation, n (%) | 6 | (30) |
| Prior systemic therapy, n (%)^{a} | | |
| Chemotherapy | 1 | (5) |
| Immune therapy (excluding PD-(L)1 inhibitor) | 3^{b} | (15) |
| Other | 2^{c} | (10) |

Footnotes:
^{a}2 subjects received multiple therapies
^{b}ipilimumab (2); cellular immunotherapy (1)
^{c}vemurafenib (1); 1 subject received MEK inhibitor & BRAF inhibitor

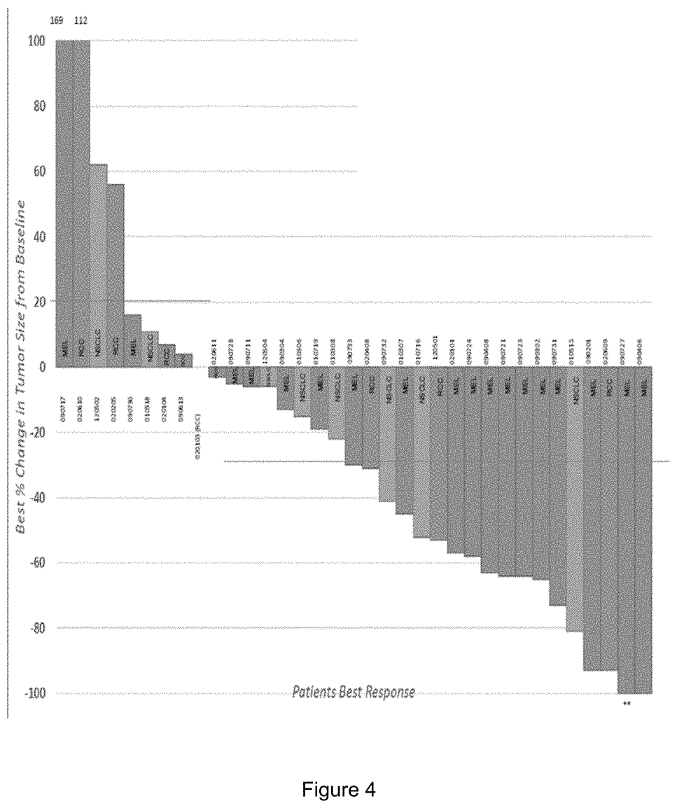

Imaging studies were performed every 8 weeks to assess tumor response according to RECIST v1.1. Tumors were observed for Objective Response Rate (ORR), Disease Control Rate (DCR), and Standard Disease (SD). The results of the imaging studies are summarized in Table 4. These results are further detailed in FIG. 4. The waterfall plot of FIG. 4 shows CPI-naïve subjects dosed with compounds of formula I in combination with nivolumab. Each bar represents a single patient's best response as defined by the sum of target lesion diameters, measured in terms of change in percent (baseline is 0% change). Bars falling within +20% increase in tumor size and −30% decrease in tumor size are considered stable disease. Further characteristics of the subjects from the study are summarized in Table 5. And the PFS characterized by metastasis distribution is summarized in Table 6 while the distribution of PFS characterized by metastatic sites is summarized in Table 7.

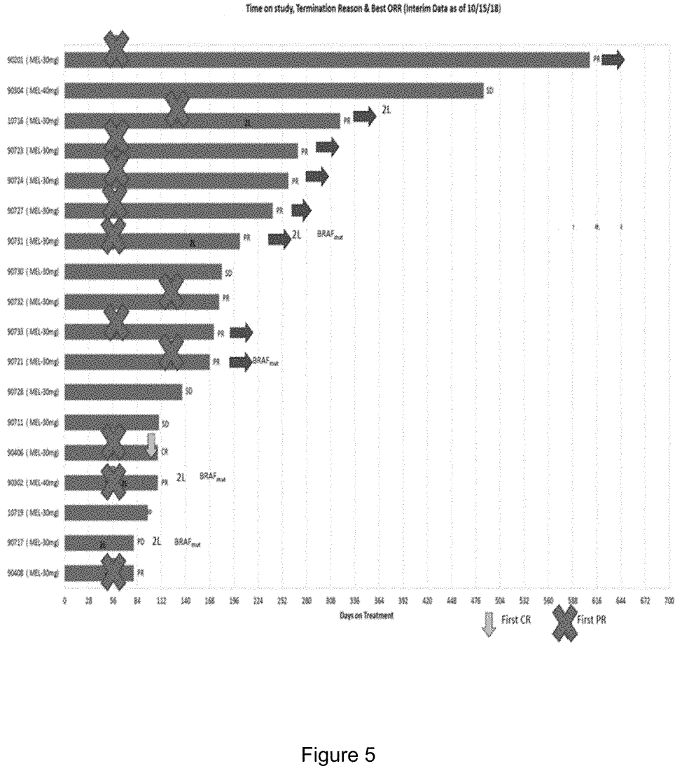

The group of MEL subjects were analyzed for total time on treatment regime, termination reason, and best ORR. The status of the subject's BRAF gene is also noted. The results are summarized in the swimmer plot of FIG. 5. All treatments were first-line unless noted with “2L.”

TABLE 4

| | Phase 1b/2 | | | |
|---|---|---|---|---|
| | MEL | RCC | NSCLC | Total |
| Enrolled | 20 | 11 | 13 | 44 |
| Evaluable | 18 | 9 | 8 | 35 |
| ORR | 67% | 33% | 38% | 51% |
| SD | 28% | 44% | 38% | 34% |
| DCR | 94% | 78% | 75% | 86% |

TABLE 5
Characteristics of Subjects

| | | | | Days | | | | | | | | |
|---|---|---|---|---|---|---|---|---|---|---|---|---|
| | | | | Since | | | | | | | Days to | |
| | | | | last | Baseline Characteristics | | | Days of Tx | | | PD | Treatment |
| | BRAF | | Prior | Syst | | M | Mets | HBI- | | Best | or | D/C |
| Subject | Mut | PD-L1 | Tx | Tx | LDH | Category | Sites | 8000 | Nivo | Response | Death | Reason |
| 090201 | | | Radiation | N/A | Normal | M1b | Lung | 669 | 687 | PR | 724+ | |
| 090408 | | | Radiation | N/A | Normal | M1b | Lung, | 81 | 57 | PR | 525+ | |
| | | | | | | | Node | | | | | |
| 010716 | Wt | Neg | Ipi | 121 | Normal | M1a | Nodes | 487+ | 487+ | PR | 487+ | |
| 090304 | | | None | N/A | Normal | M1b | Lung | 459 | 419 | SD | 459+ | |
| 090724 | Wt | | None | N/A | Normal | M1c | Pancreas | 427+ | 427+ | PR | 427+ | |
| 090723 | Pos | | Excision | N/A | Normal | M1b | Lung, | 40 | 253 | PR | 383+ | |
| | | | | | | | Node | | | | | |
| 090731 | Wt | | None | N/A | Normal | M1a | Nodes | 361+ | 361+ | PR | 361+ | |
| 090727 | Wt | | None | N/A | Normal | M1b | Lung | 109 | 336 | CR | 355+ | |
| 090721 | | | None | N/A | High | M1c | Liver, | 169 | 141 | PR | 343+ | |
| | | | | | | | Lung | | | | | |
| 090733 | V600E | | None | N/A | Normal | M1b | Lung | 341+ | 341+ | CR | 341+ | |
| 090732 | | | None | N/A | Normal | M1c | Lung, | 187 | 295 | PR | 292+ | PI decision |
| | | | | | | | Adrenal, | | | | | |
| | | | | | | | Node | | | | | |
| 010719 | | unknown | None | N/A | Normal | M1c | Liver, | 97 | 169 | SD | 197 | Clin PD |
| | | | | | | | Lung, | | | | | |
| | | | | | | | Nodes | | | | | |
| 090406 | | | Radiation | N/A | Normal | M1a | Muscle | 109 | 46 | CR | 178 | PI |
| | | | | | | | | | | | | discretin, |
| | | | | | | | | | | | | PD |
| | | | | | | | | | | | | date = death |
| 090722 | | | None | N/A | High | M1b | Lung | 31 | 169 | SD | 168+ | not |
| | | | | | | | | | | | | included in |
| | | | | | | | | | | | | ORR due |
| | | | | | | | | | | | | to <8 |
| | | | | | | | | | | | | weeks of |
| | | | | | | | | | | | | HBI-8000 |
| 090711 | | | Surg | N/A | Normal | M1c | Liver | 110 | 183 | SD | 168 | |
| | | | Resection | | | | | | | | | |
| 090730 | Pos | | None | N/A | Normal | M1a | Soft | 163 | 169 | SD | 163 | D/C due to |
| | | | | | | | tissue | | | | | clinical |
| | | | | | | | | | | | | progression |
| 090728 | Pos | | None | N/A | Normal | M1b | Lung, | 137 | 141 | SD | 137 | D/C due to |
| | | | | | | | Soft | | | | | clinical |
| | | | | | | | tissue | | | | | progression |
| 090302 | | | vemuraf/ | 30 | Normal | M1b | Lung | 109 | 127 | PR | 110 | |
| | | | TIL | | | | | | | | | |
| 090717 | Pos | | TKI | 334 | Normal | M1b | Lung | 81 | 85 | PD | 57 | |

TABLE 6
PFS by Metastasis Distribution

| PFS | | | | |
|---|---|---|---|---|
| (days) | N = 19 | M1a | M1b | M1c |
| >400 | n = 5 | 1 | 3 | 1 |
| 200-400 | n = 6 | 1 | 3 | 2 |
| 100-200 | n = 7 | 2 | 3 | 2 |
| <100 | n = 1 | | 1 | |

TABLE 7
Distribution of PFS by Metastatic Sites

| | | PFS Days | | | |
|---|---|---|---|---|---|
| M Stage | N | >400 | 200-400 | 100-200 | <100 |
| M1a | 4 | 1 | 1 | 2 | 0 |
| M1b | 10 | 3 | 3 | 3 | 1 |
| M1c | 5 | 1 | 1 | 2 | 0 |

### Example 3

Additional studies were conducted to test the effect of a combination therapy comprising compounds of formula I and nivolumab in melanoma patients with prior immune checkpoint treatment. In these studies, 8 patients were evaluable. 2 of the 8 patients showed PR. 4 of the 8 patients should SD. Further, the ORR was 25% in this group, while the DCR was 75%. A patient from this study had a tumor that was NRAS positive. Further, the patient had high LDH and an unknown level of PD-L1 expression. This patient had extensive prior treatment including surgery, radiation, ipilimumab+nivolumab, nivolumab maintenance, T-vec and pembrolizumab, TIL+high dose IL-2. This patient achieved a PR in 54 days and was on treatment for over 249 days. This PR is suggestive of epigenetic effects on the tumor. The characteristics and outcome summaries for the patients in this study are summarized in Table 8. Based on these results, combined with the data collected from melanoma-naïve patients (FIG. 5), it is recommended that the combination treatment of the compounds of formula I and nivolumab is used as a second-line treatment for patients having failed BRAF/MEK inhibitors (in patients with a BRAF mutant).

TABLE 8
Patients Characteristics and Outcome Summary-Checkpoint Inhibitor Treated

| | | | Prior | Days | | | | | | | Days | |
|---|---|---|---|---|---|---|---|---|---|---|---|---|
| | | | Systemic | Since | | | | | | | to | |
| | | | Tx | Last | Baseline Characteristics | | | Days of Tx | | | PD | Treatment |
| | BRAF | PD- | (best | Syst | | M | Mets | HBI- | | Best | or | D/C |
| Subject | Mut | L1 | response) | Tx | LDH | Category | Sites | 8000 | Nivo | Response | Death | Reason |
| 010717 | Wt | | Ipi × 4 (PR) | N/A | Normal | M1a | Node, | 98 | 112 | SD | 112 | Clinical |
| | | | Pembro × | | | | soft | | | | | progression. |
| | | | 29 (SD) | | | | tissue | | | | | Died of |
| | | | | | | | | | | | | CHF in |
| | | | | | | | | | | | | hospice |
| 090720 | V600R | | Ipi (PD) | N/A | Normal | M1c | Liver, | 55 | 57 | PD | 55 | |
| | | | Pembro (PD) | | | | Node | | | | | |
| | | | Dabra/Tram | | | | | | | | | |
| | | | (PD) | | | | | | | | | |
| | | | Fludarabine/ | | | | | | | | | |
| | | | cyclophospamide/ | | | | | | | | | |
| | | | IL-2 (PD) | | | | | | | | | |
| | | | Vemur/cobi | | | | | | | | | |
| | | | (toxicity) | | | | | | | | | |
| 090729 | V600E | | Adj | 8 months | Normal | M1a | Soft | 444+ | 444+ | PR | 444+ | |
| | | | Interferon | | | | tissue | | | | | |
| | | | TIL | | | | | | | | | |
| | | | Vemurafenib | | | | | | | | | |
| | | | Neoadj | | | | | | | | | |
| | | | Pembro | | | | | | | | | |
| 090737 | Wt | | Adj | 4 months | High | M1a | Renal | 199 | 209 | PR | 358+ | |
| | | | Interferon | | | | hilar | | | | | |
| | | | Nivo/ipi + | | | | mass | | | | | |
| | | | nivo | | | | Pelvic | | | | | |
| | | | T-Vec | | | | mass | | | | | |
| | | | Pembro | | | | Sub-cu | | | | | |
| | | | TIL + high | | | | mass | | | | | |
| | | | dose | | | | node | | | | | |
| | | | IL-2 | | | | | | | | | |
| 090738 | V600K | | Dabra/Tram | 14 months | Normal | M1c | Nodes, | 201 | 225 | SD | 243 | Death not |
| | | | Pembro 2 | | | | liver | | | | | related to |
| | | | years | | | | | | | | | study |
| | | | Ipi/pembro × | | | | | | | | | treatment |
| | | | 4 | | | | | | | | | |
| | | | Pembro | | | | | | | | | |
| 090739 | unknown | | Pembro | N/A | Normal | M1a | Soft | 103 | 113 | SD | 133+ | Pt withdrew |
| | | | (SD) | | | | tissue | | | | | from study |
| | | | Pembro + ipi | | | | nodules | | | | | to pursue |
| | | | (SD) | | | | | | | | | other |
| | | | | | | | | | | | | treatment |

### Example 4

The tolerability of the compounds of formula I, nivolumab, and a combination of the compounds of formula I and nivolumab were tested. The Phase 2 clinical dosage of 30 mg BIW of the compounds of formula I was administered. Among 63 subjects with adverse event (“AE”) data available, Treatment Emergent Adverse Event (“TEAE”), 52% were considered related to treatment (“TRAE”). Among TRAEs, 47% were associated with the combination of the compounds of formula I and nivolumab (“NIVO”), 39% compounds of formula I alone, and 14% NIVO alone. Less frequent TRAEs associated with NIVO alone were lipase increase (n=6), rash (n=10), TSH increase (n=5), amylase increase (n=4). Further, among Grade 3 AEs, only fatigue, headache, diarrhea, nausea, vomiting were symptomatic—others were asymptomatic. Grade 3 fatigue one subject responded to a low dose oral steroid. The fatigue another subject was resolved by withholding drug only—no intervention necessary. Grade 3 diarrhea was resolved with over counter drug. Grade 3 headache responded to over counter drug and did not recur on subsequent dosings. And nausea and vomiting responded to oral drug. None was difficult to manage in oncology practice, and none caused medical concerns by investigators. Other asymptomatic AEs were abnormalities of blood test, most did not require treatments. When counting AEs with symptoms, only 4 out of 20 subjects experienced clinically significant AEs on nivo+compounds of formula I. The findings of this study are summarized in Table 9 below.

TABLE 9
Tolerability Profile HBI-8000 + NIVO
TRAE ≥ 5% frequency and number of subject n (% of N)

| | Association | | | | | | | | |
|---|---|---|---|---|---|---|---|---|---|
| | Compounds of Formula I + | | | | | | | | |
| AE | NIVO | | | Compounds of Formula I | | | NIVO | | |
| Term | G1-2 | G3 | G4 | G1-2 | G3 | G4 | G1-2 | G3 | G4 |
| Fatigue | 24 (38) | 4 (6) | 0 | 0 | 0 | 0 | 0 | 0 | 0 |
| Diarrhea | 19 (30) | 3 (5) | 0 | 0 | 0 | 0 | 4 (6) | 1 (2) | 0 |
| Lymphocyte | 6 (10) | 1 (2) | 0 | 0 | 0 | 0 | 0 | 0 | 0 |
| decrease | | | | | | | | | |
| Neutrophil | 4 (6) | 2 (3) | 0 | 8 (13) | 5 (8) | 1 (2) | 0 | 0 | 0 |
| decrease | | | | | | | | | |
| WBC decrease | 6 (10) | 1 (2) | 0 | 11 (17) | 1 (2) | 0 | 0 | 0 | 0 |
| Platelet decrease | 10 (16) | 0 | 0 | 23 (37) | 3 (5) | 0 | 0 | 0 | 0 |
| Anemia | 0 | 0 | 0 | 14 (22) | 1 (2) | 0 | 0 | 0 | 0 |
| Hypophospatemia | 0 | 0 | 0 | 6 (10) | 4 (6) | 0 | 0 | 0 | 0 |

### Example 5

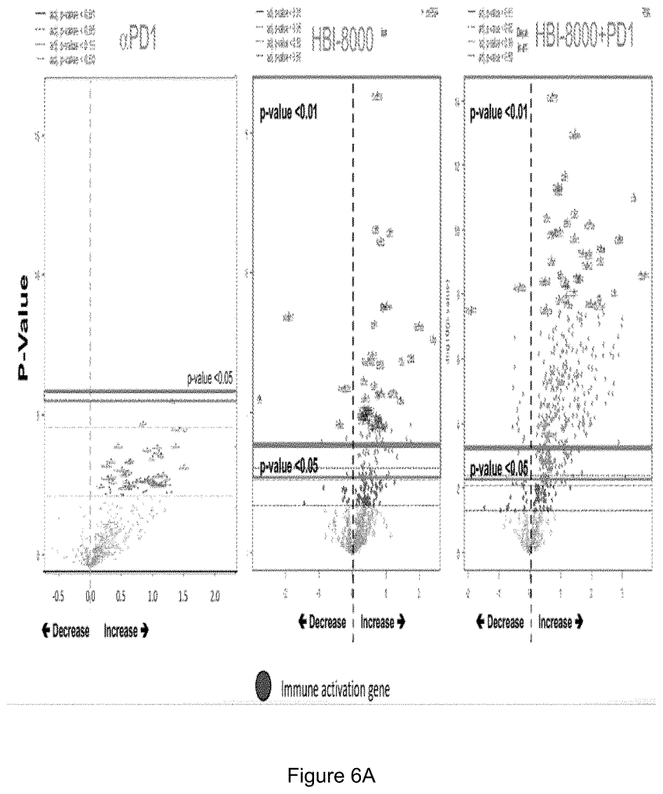

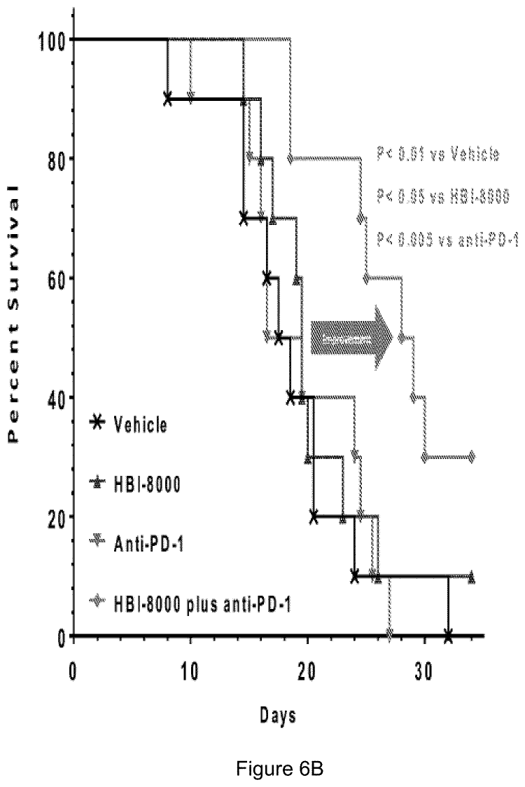

Immune gene activation in response to administration of the compounds of formula I, a PD-1 inhibitory antibody, and a combination of the compounds of formula I and a PD-1 inhibitory antibody was examined using an MC38 tumor model. The results are summarized in FIG. 6A. These results show that the combination of the compounds of formula I and a PD-1 inhibitory antibody synergistically activated immune gene expression in the tumor microenvironment. FIG. 6B summarizes the improvement on survival amongst the experimental group treated with the combination therapy compared to the compounds of formula I alone or the PD-1 inhibitory antibody alone.

### Example 6

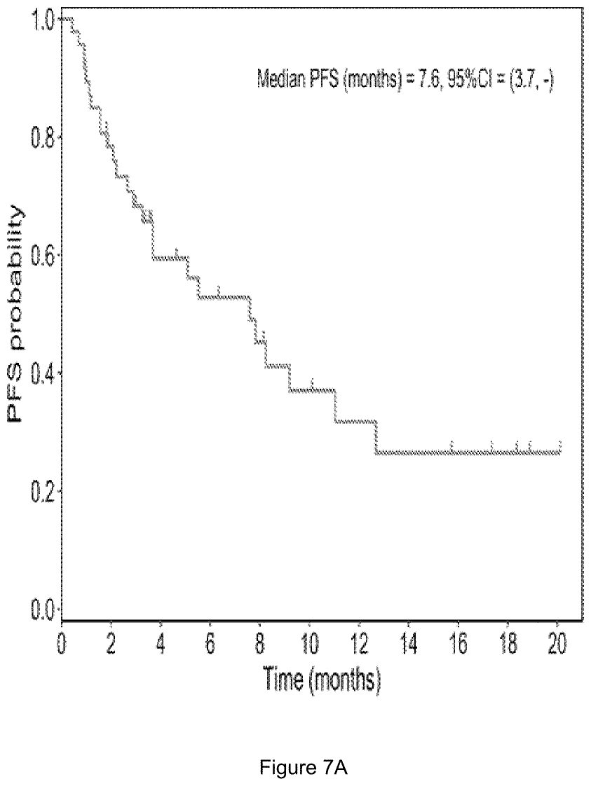

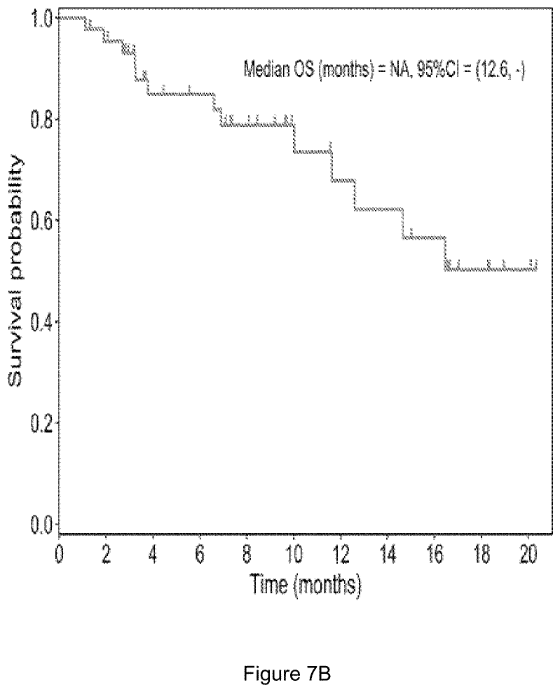

The compounds of formula I were used as a monotherapy for relapsed or refractory peripheral T-cell lymphoma (“RR/PTCL”). A dosage of 40 mg biw was approved for this study. A summary of the results is found in Table 10. These results were compared to efficacy/history benchmarks for several known treatments for RR/PTCL (see Table 11). The time after prior treatment was 96 days (vs. 222 days for romidepsin P2 trial). The estimated PFS and OS of the experiment are found in FIGS. 7A-B. For these results, the PD date was due to investigator judgement. The median PFS (months) was 7.6.

TABLE 10
Example 6 Results Summary

| Patients | Numbers |
|---|---|
| Phase 2 Enrolled Total | 55 |
| Evaluable Total | 43 |
| Complete Responses | 4 |
| Partial Responses | 16 |
| Stable Disease | 12 |
| ORR | 46% |
| DCR | 74% |
| Pharmaceuticals and Medical Devices | 30% |
| Agency (“PMDA”) Target ORR | |

TABLE 11
Efficacy vs. Historical Benchmarks for RR/PTCL Treatments

| RR/PTCL Treatment | ORR |
|---|---|
| Epidaza (China) (PMS) | 28% (35%) |
| Forodesine | 22% |
| Pralatrexate | 45% |
| Romidepsin | 43% |

### Example 7

The compounds of formula I were used as a monotherapy for relapsed or refractory aggressive adult T-cell lymphoma (“RR/ATL”). A dosage of 40 mg biw was approved for this study. A summary of the results is found in Table 12. These results were compared to efficacy/history benchmarks for several known treatments for RR/ATL (see Table 13). The time after prior treatment was 88 days (vs. 234 days for lenalidomide P2 trial). All patients had received mogamulizumab (“moga”) (lenalidomide ORR in moga patients was 18%).

TABLE 12
Example 7 Results Summary

| Patients | Numbers |
|---|---|
| Phase 2 Enrolled Total | 23 |
| Evaluable Total | 20 |
| Partial Responses | 8 |
| Stable Disease | 4 |
| ORR | 40% |
| DCR | 60% |
| Pharmaceuticals and Medical Devices | 30% |
| Agency (“PMDA”) Target ORR | |

TABLE 13
Efficacy vs. Historical Benchmarks for RR/ATL Treatments

| RR/PTCL Treatment | ORR |
|---|---|
| Compounds of Formula I Phase I | 75% |
| Mogamulizumab | 50% |
| Lenalidomide | 42% |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A combination comprising a therapeutically effective amount of a PD-L1 inhibitor, a PD-1 inhibitor, a CTLA-4 inhibitor, a CD276 inhibitor, a therapeutically effective amount of a compound of formula I, or pharmaceutically acceptable salt or solvate thereof, or any combination thereof, wherein formula I is: 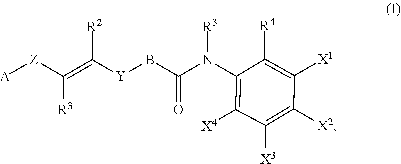
wherein,
A is phenyl or a heterocyclic group, optionally substituted with 1 to 4 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkythio, C₁-C₄ perfluoroalkyl, C₁-C₄ perfluoroalkyloxy, C₁-C₄ alkoxycarbonyl, phenyl, and a heterocyclic group;
B is phenyl optionally substituted with 1 to 3 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, C₁-C₄ alkoxycarbonyl, and phenyl;
Y is a moiety comprising —CO— which is linear and in which the distances between the centroid of ring B (W1), the centroid of ring A (W2) and an oxygen atom as a hydrogen bond acceptor in the moiety Y (W3) are: W1-W2=about 6.0 Å, W1-W3=about 3.0 Å to about 6.0 Å, and W2-W3=about 4.0 Å to about 8.0 Å, respectively;
Z is a bond or C₁-C₄ alkylene, —O—, —S—, —NH—, —CO—, —CS—, —SO—, or —SO₂—;
R¹ and R² are independently hydrogen or C₁-C₄ alkyl;
R³ is hydrogen or C₁-C₄ alkyl;
R⁴ is hydrogen or —NH₂;
one of X¹, X², X³, or X⁴ is halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, or C₁-C₄ alkoxycarbonyl optionally substituted with halogen or C₁-C₄ alkyl, while the others of X¹, X², X³, or X⁴ are independently hydrogen,
provided that when R⁴ is hydrogen, one of X¹, X², X³, or X⁴ is —NH₂, an aminoalkyl group or an alkylamino group.

2. The combination of claim 1, wherein said compound of formula I has the structure of formula Ia: 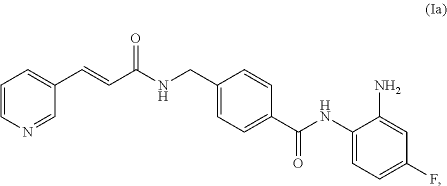 [SMILES: Nc1cc(F)ccc1NC(=O)c1ccc(CNC(=O)/C=C/c2cccnc2)cc1]
or a pharmaceutically acceptable salt or solvate thereof.

3. The combination of claim 1, wherein said compound of formula I is N-(2-amino-4-fluorophenyl)-4-[[[(2E)-1-oxo-3-(3-pyridinyl)-2-propen-1-yl]amino]methyl]benzamide.

4. The combination of claim 1, wherein said PD-L1 inhibitor, PD-1 inhibitor, CTLA-4 inhibitor, and/or CD276 inhibitor is a small molecule compound, a nucleic acid, a peptide, a protein, an antibody, a peptibody, a diabody, a minibody, a single-chain variable fragment (ScFv), or a fragment or variant thereof.

5. The combination of claim 4, wherein at least one of said PD-L1 inhibitor, PD-1 inhibitor, CTLA-4 inhibitor, and/or CD276 inhibitor is an antibody.

6. The combination of claim 5, wherein said inhibitor antibody is a monoclonal antibody.

7. The combination of claim 5 or 6, wherein said inhibitor antibody comprises a human antibody, a mouse antibody, a chimeric antibody, a humanized antibody, or a chimeric humanized antibody.

8. The combination of one of claims 5 to 7, wherein said inhibitor antibody is a human antibody or a humanized antibody.

9. The combination of one of claims 5 to 8, wherein said inhibitor antibody is present at an amount of about 0.1 mg/kg to about 30 mg/kg.

10. The combination of one of claims 5 to 9, wherein said inhibitor antibody is present at an amount of about 0.5 mg/kg to about 15 mg/kg.

11. The combination of one of claims 5 to 10, wherein said inhibitor antibody is present at an amount of about: 0.1 mg/kg, 0.3 mg/kg, 1 mg/kg, 2 mg/kg, 2.5 mg/kg, 3 mg/kg, 5 mg/kg, 10 mg/kg, or 20 mg/kg.

12. The combination of any one of claims 1 to 11, wherein said combination is suitable for parenteral administration to a cancer patient.

13. The combination of claim 12, wherein said parenteral administration comprises intravenous (IV) administration.

14. A pharmaceutical composition comprising a combination of any one of claims 1 to 13 and a pharmaceutically acceptable excipient.

15. A kit comprising the combination of any of one of claims 1 to 13 or a pharmaceutical composition of claim 14.

16. The kit of claim 15, further comprising at least one administration device.

17. The kit of claim 15 or 16, wherein components in the kit are sterilized.

18. A method for treating cancer, said method comprising administering a therapeutically effective amount of a combination of any one of claims 1 to 13 or a pharmaceutical composition of claim 14 to a subject in need thereof.

19. The method of claim 18, wherein said subject has a mutated BRAF gene.

20. The method of claim 18 or 19, wherein said cancer is a solid tumor cancer selected from the group consisting of squamous cell carcinoma, nonsquamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, colorectal cancer, pancreatic cancer, lymphoma, leiomyosarcoma, liposarcoma, synovial sarcoma, or malignant peripheral sheath tumor (MPNST).

21. The method of claim 20, wherein said cancer is non-small cell lung cancer (NSCLC), hepatocellular carcinoma, melanoma, ovarian cancer, breast cancer, pancreatic cancer, renal cell carcinoma, or colorectal cancer.

22. The method of claim 20, wherein said cancer is lymphoma, Non-Hodgkin's lymphoma (NHL), Hodgkin's Lymphoma, Reed-Sternberg disease, multiple myeloma (MM), acute myelogenous leukemia (AML), chronic myelogenous leukemia (CIVIL), acute lymphocytic leukemia, (ALL), or chronic lymphocytic leukemia (CLL).

23. The method of any one of claims 18 to 21, wherein said cancer patient is treatment naïve.

24. The method of claim 22, wherein said cancer patient is treatment naïve for non-small cell lung cancer (NSCLC), hepatocellular carcinoma, melanoma, ovarian cancer, breast cancer, pancreatic cancer, renal cell carcinoma, or colorectal cancer.

25. The method of any one of claims 18 to 24, wherein said combination is administered to said cancer patient as a first line therapy.

26. The method of any one of claims 18 to 24, wherein said combination is administered to said cancer patient as a second, third, fourth, fifth, or sixth line of treatment.

27. The method of any one of claims 18 to 24, wherein said combination is administered to said cancer patient following treatment with at least one anti-cancer therapy.

28. The method of claim 27, wherein said anti-cancer therapy comprises chemotherapy, radiotherapy, surgery, targeted therapy, immunotherapy, or a combination thereof.

29. The method of any one of claims 18 to 28, wherein said cancer is resistant to at least one anti-cancer agent.

30. The method of any one of claims 18 to 29, wherein said compound of formula I and said inhibitor of said combination are administered simultaneously or sequentially.

31. The method of any one of claims 18 to 30, wherein said compound of formula I is administered 2 to 3 times per week.

32. The method of any one of claims 18 to 31, wherein said compound of formula I is administered daily.

33. The method of any one of claims 18 to 32, wherein said PD-L1 inhibitor, PD-1 inhibitor, CTLA-4 inhibitor, and/or CD276 inhibitor and said compound of formula I are concomitantly administered on day 1 of an administration regimen.

34. The method of claims 18 to 33, wherein said combination is administered to said patient as a regimen.

35. The method of claim 34, wherein said regimen is repeated until disease progression or unacceptable toxicity.

36. The method of claim 34, wherein said regimen comprises a rest period of at least 1 day between consecutive administration periods.

37. The method of any one of claims 18 to 36, wherein said compound of formula I of said combination is administered 2 to 3 times per week in said regimen and said PD-L1 inhibitor, PD-1 inhibitor, CTLA-4 inhibitor, and/or CD276 inhibitor is administered every 2 to 3 weeks.

38. The method of claim 37, wherein said compound of formula I of said combination is administered once a day (“QD”) for 21 days in said regimen and said inhibitor antibody is administered every 2 to 3 weeks.

39. The method of any one of claims 18 to 38, wherein said method of treating cancer inhibits metastasis of said cancer in said patient.

40. The method of any one of claims 18 to 39, wherein said method of treating cancer reduces tumor or tumor burden in said patient.

41. The method of any one of claims 18 to 40, wherein said method of treating cancer inhibits pre-existing metastasis of said cancer in said patient.

42. The method of any one of claims 18 to 41, wherein said method of treating cancer prolongs the time to disease progression of said cancer in said patient.

43. The method of any one of claims 18 to 42, wherein said method of treating cancer prolongs the survival of said patient.

44. The method of any one of claims 18 to 43, wherein said method of treating cancer increases progression-free survival of said patient.

45. A method for treating cancer comprising administering a therapeutically effective amount of a combination of a histone deacetylase inhibitor (HDAC inhibitor) and a PD-L1 inhibitor and/or a PD-1 inhibitor, plus a CTLA-4 inhibitor, to a subject in need of treatment and whose cancer has been previously treated with a checkpoint inhibitor.

46. A method for treating cancer comprising administering a therapeutically effective amount of a PD-L1 inhibitor, a PD-1 inhibitor, a CTLA-4 inhibitor, a CD276 inhibitor, a histone deacetylase inhibitor (HDAC inhibitor), or any combination thereof, to a subject in need of treatment and whose cancer has been previously treated with a checkpoint inhibitor.

47. A method for treating cancer comprising administering a therapeutically effective amount of a PD-L1 inhibitor, a PD-1 inhibitor, a CTLA-4 inhibitor, a CD276 inhibitor, a histone deacetylase inhibitor (HDAC inhibitor), or any combination thereof, to a subject in need of treatment wherein said subject comprises a mutated BRAF gene.

48. A method for treating cancer comprising administering a therapeutically effective amount of:
(a) a compound of formula I, or a pharmaceutically acceptable salt or solvate thereof; wherein formula I is: 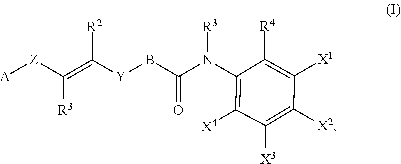
wherein,
A is phenyl or a heterocyclic group, optionally substituted with 1 to 4 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkythio, C₁-C₄ perfluoroalkyl, C₁-C₄ perfluoroalkyloxy, C₁-C₄ alkoxycarbonyl, phenyl, and a heterocyclic group;
B is phenyl optionally substituted with 1 to 3 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, C₁-C₄ alkoxycarbonyl, and phenyl;
Y is a moiety comprising —CO— which is linear and in which the distances between the centroid of ring B (W1), the centroid of ring A (W2) and an oxygen atom as a hydrogen bond acceptor in the moiety Y (W3) are: W1-W2=about 6.0 Å, W1-W3=about 3.0 Å to about 6.0 Å, and W2-W3=about 4.0 Å to about 8.0 Å, respectively;
Z is a bond or C₁-C₄ alkylene, —O—, —S—, —NH—, —CO—, —CS—, —SO—, or —SO₂—;
R¹ and R² are independently hydrogen or C₁-C₄ alkyl;
R³ is hydrogen or C₁-C₄ alkyl;
R⁴ is hydrogen or —NH₂;
one of X¹, X², X³, or X⁴ is halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, or C₁-C₄ alkoxycarbonyl optionally substituted with halogen or C₁-C₄ alkyl, while the others of X¹, X², X³, or X⁴ are independently hydrogen, provided that when R⁴ is hydrogen, one of X¹, X², X³, or X⁴ is —NH₂, an aminoalkyl group or an alkylamino group; and
(b) one or more inhibitor antibodies, wherein said one or more inhibitor antibodies comprise a PD-L1 inhibitor, a PD-1 inhibitor, a CTLA-4 inhibitor, a CD276 inhibitor, or any combination thereof, and wherein said one or more inhibitor antibodies are present at an amount of about 0.1 mg/kg to about 30 mg/kg;
to a subject in need of treatment.

49. The method of claim 48, wherein said compound of formula I, has the structure of formula Ia. 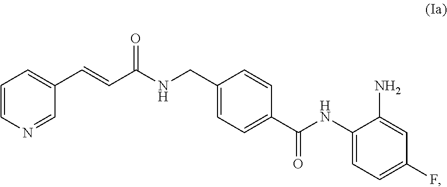 [SMILES: Nc1cc(F)ccc1NC(=O)c1ccc(CNC(=O)/C=C/c2cccnc2)cc1]
or a pharmaceutically acceptable salt or solvate thereof.

50. The method of claim 48, wherein said compound of formula I is N-(2-amino-4-fluorophenyl)-4-[[[(2E)-1-oxo-3-(3-pyridinyl)-2-propen-1-yl]amino]methyl]benzamide.

51. The method of any one of claims 48 to 50, wherein said one or more inhibitor antibodies are monoclonal antibodies.

52. The method of any one of claims 48 to 51, wherein said one or more inhibitor antibodies comprise a human antibody, a mouse antibody, a chimeric antibody, a humanized antibody, or a chimeric humanized antibody.

53. The method of any one of claims 48 to 52, wherein said inhibitor antibody is a human antibody or a humanized antibody.

54. The method of any one of claims 48 to 53, wherein said cancer is a solid tumor cancer selected from the group consisting of squamous cell carcinoma, nonsquamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, colorectal cancer, pancreatic cancer, lymphoma, leiomyosarcoma, liposarcoma, synovial sarcoma, or malignant peripheral sheath tumor (MPNST).

55. The method of any one of claims 48 to 54, wherein said cancer patient is treatment naïve.

56. The method of claim 55, wherein said cancer patient is treatment naïve for non-small cell lung cancer (NSCLC), hepatocellular carcinoma, melanoma, ovarian cancer, breast cancer, pancreatic cancer, renal cell carcinoma, or colorectal cancer.

57. The method of any one of claims 48 to 56, wherein said combination is administered to said cancer patient as a first line therapy.

58. The method of any one of claims 48 to 56, wherein said combination is administered to said cancer patient as a second, third, fourth, fifth, or sixth line of treatment.

59. The method of any one of claims 48 to 56, wherein said combination is administered to said cancer patient following treatment with at least one anti-cancer therapy.

60. The method of claim 59, wherein said anti-cancer therapy comprises chemotherapy, radiotherapy, surgery, targeted therapy, immunotherapy, or a combination thereof.

61. The method of any one of claims 48 to 60, wherein said cancer is resistant to at least one anti-cancer agent.

62. The method of any one of claims 48 to 61, wherein said compound of formula I and said inhibitor of said combination are administered simultaneously or sequentially.

63. The method of any one of claims 48 to 62, wherein said compound of formula I is administered 2 to 3 times per week.

64. The method of any one of claims 48 to 63, wherein said compound of formula I is administered daily.

65. The method of any one of claims 48 to 64, wherein said PD-L1 inhibitor, PD-1 inhibitor, CTLA-4 inhibitor, and/or CD276 inhibitor and said compound of formula I are concomitantly administered on day 1 of an administration regimen.

66. The method of any one of claims 48 to 64, wherein said PD-L1 inhibitor, PD-1 inhibitor, CTLA-4 inhibitor, and/or CD276 inhibitor and said compound of formula I are administered to said patient as a regimen.

67. The method of claim 66, wherein said regimen is repeated until disease progression or unacceptable toxicity.

68. The method of claim 66, wherein said regimen comprises a rest period of at least 1 day between consecutive administration periods.

69. The method of any one of claims 48 to 68, wherein said compound of formula I is administered 2 to 3 times per week and said PD-L1 inhibitor, PD-1 inhibitor, CTLA-4 inhibitor, and/or CD276 inhibitor is administered every 2 to 3 weeks.

70. The method of claim 69, wherein said compound of formula I of said combination is administered once a day (“QD”) for 21 days in said regimen and said inhibitor antibody is administered every 2 to 3 weeks.
